(19) United States
(12) Patent Application Publication
(10) Pub. No.: US 2024/0312558 A1
(43) Pub. Date: Sep. 19, 2024

(54) MACHINE LEARNING SYSTEMS AND METHODS FOR DEEP LEARNING OF GENOMIC CONTEXTS

(71) Applicant: President and Fellows of Harvard College, Cambridge, MA (US)

(72) Inventors: Yunha Hwang, Cambridge, MA (US); Sergey Ovchinnikov, Cambridge, MA (US)

(21) Appl. No.: 18/605,451

(22) Filed: Mar. 14, 2024

Related U.S. Application Data
(60) Provisional application No. 63/491,019, filed on Mar. 17, 2023.

(51) Int. Cl. G16B 20/00; G06N 3/08; G16B 40/20; G16B 45/00

(52) U.S. Cl. CPC G16B 20/00; G06N 3/08; G16B 40/20; G16B 45/00

(57) ABSTRACT

Some aspects provide for a method for generating a contextual embedding of a gene. In some embodiments, the method comprises: using at least one computer hardware processor to perform: obtaining information specifying genomic context of the gene, the genomic context containing a plurality of genes including the gene, the information containing gene sequences for the plurality of genes; encoding the information specifying the genomic context to obtain an initial encoding of the genomic context, the encoding comprising: mapping the gene sequences to protein sequences; and encoding the protein sequences using a trained protein language model (pLM) to obtain the initial encoding of the genomic context; and processing the initial encoding of the genomic context with a genomic language model (gLM) to obtain the contextual embedding of the gene.

### RELATED APPLICATIONS

This application claims the benefit of priority, under 35 U.S.C. § 119(e), to U.S. Application Ser. No. 63/491,019, filed Mar. 17, 2023, entitled “TECHNIQUES FOR DEEP LEARNING OF GENOMIC CONTEXTS,” the entire contents of which are incorporated by reference herein.

### BACKGROUND

DNA includes genes and intergenic regions. Genes can include protein-coding genes and non-coding genes. Intergenic regions are sequences of the DNA that are located between genes.

### SUMMARY

Some aspects provide for a method for generating a contextual embedding of a gene. In some embodiments, the method comprises: using at least one computer hardware processor to perform: obtaining information specifying genomic context of the gene, the genomic context containing a plurality of genes including the gene, the information containing gene sequences for the plurality of genes; encoding the information specifying the genomic context to obtain an initial encoding of the genomic context, the encoding comprising: mapping the gene sequences to protein sequences; and encoding the protein sequences using a trained protein language model (pLM) to obtain the initial encoding of the genomic context; and processing the initial encoding of the genomic context with a genomic language model (gLM) to obtain the contextual embedding of the gene.

Some aspects provide for a system, comprising: at least one computer hardware processor; and at least one non-transitory computer-readable storage medium storing processor-executable instructions that, when executed by the at least one computer hardware processor, cause the at least one computer hardware processor to perform a method for generating a contextual embedding of a gene. In some embodiments, the method comprises: using at least one computer hardware processor to perform: obtaining information specifying genomic context of the gene, the genomic context containing a plurality of genes including the gene, the information containing gene sequences for the plurality of genes; encoding the information specifying the genomic context to obtain an initial encoding of the genomic context, the encoding comprising: mapping the gene sequences to protein sequences; and encoding the protein sequences using a trained protein language model (pLM) to obtain the initial encoding of the genomic context; and processing the initial encoding of the genomic context with a genomic language model (gLM) to obtain the contextual embedding of the gene.

Some aspects provide for at least one non-transitory computer-readable storage medium storing processor-executable instructions that, when executed by at least one computer hardware processor, cause the at least one computer hardware processor to perform a method for generating a contextual embedding of a gene. In some embodiments, the method comprises: using at least one computer hardware processor to perform: obtaining information specifying genomic context of the gene, the genomic context containing a plurality of genes including the gene, the information containing gene sequences for the plurality of genes; encoding the information specifying the genomic context to obtain an initial encoding of the genomic context, the encoding comprising: mapping the gene sequences to protein sequences; and encoding the protein sequences using a trained protein language model (pLM) to obtain the initial encoding of the genomic context; and processing the initial encoding of the genomic context with a genomic language model (gLM) to obtain the contextual embedding of the gene.

In some embodiments, the genomic context is a gene subcontig containing the plurality of genes.

In some embodiments, the genomic context consists of 10-50 genes.

In some embodiments, the genomic context consists of 15-30 genes.

In some embodiments, mapping the gene sequences to protein sequences comprises identifying for each of the gene sequences a representative protein sequence.

In some embodiments, the pLM is an ESM2 protein language model.

In some embodiments, the genomic context comprises the plurality of genes and a plurality of intergenic regions, the information containing intergenic sequences for the plurality of intergenic regions, and encoding the information specifying the genomic context further comprises: encoding the protein sequences and the intergenic sequences to obtain the initial encoding of the genomic context, the initial encoding comprising representations of the protein sequences and representations of the intergenic sequences.

In some embodiments, encoding the protein sequences and the intergenic sequences to obtain the initial encoding of the genomic context comprises: encoding the protein sequences using the trained pLM to obtain the representations of the protein sequences; and encoding the intergenic sequences using a trained intergenic sequence model to obtain the representations of the intergenic sequences.

In some embodiments, the genomic context includes K genes and the information includes K gene sequences; mapping the gene sequences to protein sequences comprises mapping the K gene sequences to K protein sequences; and encoding the protein sequences comprises encoding each of the protein sequences as an N-dimensional vector such that the initial encoding of the genomic context comprises K N-dimensional vectors.

In some embodiments, K is between 15 and 30, inclusive, and wherein N is between 800 and 1600.

In some embodiments, the genomic language model comprises a multi-layer transformer model.

In some embodiments, the contextual embedding of the gene is obtained from hidden states of the genomic language model.

In some embodiments, the contextual embedding of the gene is obtained from the last hidden states of the genomic language model.

In some embodiments, the genomic language model comprises multiple hidden layers and multiple attention heads per layer.

In some embodiments, the genomic language model comprises 15-25 hidden layers and between 5-15 attention heads per hidden layer.

Some embodiments further comprise: using the contextual embedding of the gene to identify a putative function to a protein corresponding to the gene.

In some embodiments, using the contextual embedding to identify the putative function comprises comparing the contextual embedding of the gene to contextual embeddings of other genes whose proteins have functional annotations.

Some embodiments further comprise: using the context embedding of the gene for annotation transfer.

In some embodiments, the gene is a microbial gene.

Some embodiments further comprise: obtaining one or more attention mappings from the gLM.

### BRIEF DESCRIPTION OF DRAWINGS

Various aspects and embodiments of the disclosure provided herein are described below with reference to the following figures. The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1A and FIG. 1B are diagrams of illustrative techniques for generating a contextual embedding of a gene, according to some embodiments of the technology described herein.

FIG. 2 is a block diagram of an example system 200 for generating a contextual embedding of a gene, according to some embodiments of the technology described herein.

FIG. 3A is a flowchart of an illustrative process 300 for generating a contextual embedding of a gene, according to some embodiments of the technology described herein.

FIG. 3B is a flowchart of an illustrative process 350 for training a genomic language model (gLM) to obtain a contextual embedding of a gene, according to some embodiments of the technology described herein.

FIG. 4A is a diagram of an example technique for training a gLM to predict a contextual embedding of a gene, according to some embodiments of the technology described herein.

FIG. 4B is a diagram of an example technique for using a trained gLM to obtain a contextual embedding of a gene, according to some embodiments of the technology described herein.

FIG. 5A and FIG. 5B are validation accuracy curves showing that a trained gLM more accurately predicts masked protein sequence representations than a bidirectional long short-term memory (LSTM) model, according to some embodiments of the technology described herein.

FIG. 6A shows that genes may have the same protein embedding, but different contextual embeddings, according to some embodiments of the technology described herein.

FIG. 6B and FIG. 6C show that the contextualized McrA embeddings cluster with the direction of a reaction that the MCR complex is likely to carry out, according to some embodiments of the technology described herein.

FIGS. 6D and 6E show contextualized protein embeddings where phage defense proteins cluster and biosynthetic gene products cluster, according to some embodiments of the technology described herein.

FIG. 7A, FIG. 7B, and FIG. 7C show contig averaged protein language model (pLM) embeddings, according to some embodiments of the technology described herein.

FIG. 8A is a diagram showing an example of how context-free, context-only, and contextualized gene embeddings are extracted, according to some embodiments of the technology described herein.

FIG. 8B and FIG. 8C compare the per-layer linear probing accuracies of gLM contextualized embeddings with the per-layer linear probing accuracies of gLM context-only embeddings, according to some embodiments of the technology described herein.

FIG. 9A, FIG. 9B, FIG. 9C, and FIG. 9D show that the genomic context of proteins can be used to improve the expressiveness of protein representations for enzyme function prediction, according to some embodiments of the technology described herein.

FIG. 10A and FIG. 10B show that attention heads in shallower layers of a trained gLM correlate with operons, according to some embodiments of the technology described herein.

FIG. 10C shows that a logistic regression classifier trained using attention patterns across attention heads of a gLM can predict the presence of an operonic pair of neighboring proteins in a sequence with high precision, according to some embodiments of the technology described herein.

FIG. 10D shows pLM-generated protein embeddings of AAA+ regulator proteins, according to some embodiments of the technology described herein.

FIG. 10E shows combined protein and context embeddings of the AAA+ regulator proteins of FIG. 10D, according to some embodiments of the technology described herein.

FIG. 11 shows a visualization of attention patterns across attention heads of a gLM for a randomly chosen sequence, according to some embodiments of the technology described herein.

FIG. 12A and FIG. 12B shows functional association predictions for the AAA+ proteins of FIG. 10D and FIG. 10E, according to some embodiments of the technology described herein.

FIG. 12C shows the gLM contextual embeddings of the AAA+ regulator proteins of FIG. 10D and FIG. 10E, according to some embodiments of the technology described herein.

FIG. 12D shows the contig-averaged pLM baseline for the AAA+ regulator proteins of FIG. 10D and FIG. 10E, according to some embodiments of the technology described herein.

FIG. 13A and FIG. 13B show the distance-based clustering of raw embeddings of AAA+ regulators and controls shown in FIG. 10D and FIG. 10E.

FIG. 14A shows ModA and ModC interactions, according to some embodiments of the technology described herein.

FIG. 14B and FIG. 14C show the performance of the gLM in predicting the embedding of interacting paralogs (e.g., ModA and ModC), according to some embodiments of the technology described herein.

FIG. 14D and FIG. 14E compare context-free contig embeddings and contextualized contig embeddings of random 30-protein subcontigs, according to some embodiments of the technology described herein.

FIG. 14F shows that the average precision of a logistic regression classifier trained on contextualized contig embeddings is greater than the average precision of a logistic regression classifier trained on context-free embeddings, according to some embodiments of the technology described herein.

FIGS. 15A and 15B show the contextualized embeddings and context-free embeddings used to train the logistic regression classifiers used to generate the curves of FIG. 14F, according to some embodiments of the technology described herein.

FIG. 15C and FIG. 15D are confusion matrices showing that the average precision of a logistic regression classifier trained on the contextualized embeddings of FIG. 15B is greater than the average precision of a logistic regression classifier trained on the context-free embeddings of FIG. 15A, according to some embodiments of the technology described herein.

FIG. 16A shows the cumulative distribution of contig lengths used for generating training data for training a gLM to predict contextual embeddings, according to some embodiments of the technology described herein.

FIG. 16B shows the cumulative variance of principal components of ESM2 embeddings, according to some embodiments of the technology described herein.

FIG. 17 is a schematic diagram of an illustrative computing device with which aspects described herein may be implemented.

### DETAILED DESCRIPTION

Evolutionary processes result in the linkage between protein sequences, structure and function. The resulting sequence-structure-function paradigm has provided the basis for interpreting vast amounts of genomic data. Protein language models (pLMs) have been used to represent these complex relationships shaped by evolution, considering each protein as an independent and standalone entity. However, proteins are encoded in genomes alongside other proteins, and the specific genomic context that a protein occurs in is determined by evolutionary processes where each gene gain, loss, duplication and transposition event is subject to selection and drift. These processes are particularly pronounced in bacterial and archaeal genomes where frequent horizontal gene transfers (HGT) shape genomic organization and diversity. Thus, there exists an inherent evolutionary linkage between genes, their genomic context, and gene function. By considering proteins independently, pLMs fail to capture these complex, contextual relationships.

While there have been some approaches to modeling genomic information by considering genomic context, there are several disadvantages associated with such conventional techniques. First, the conventional techniques are limited in accuracy and reliability because they represent genes as categorical entities, despite these genes existing in continuous space where multidimensional properties such as phylogeny, structure, and function are abstracted in their sequences. By failing to account for these properties, the conventional techniques are limited in their accuracy and reliability. Second, the conventional techniques lack generalizability because they are trained on short genomic segments from narrow lineages of organisms and fail to represent genes in continuous space.

Accordingly, the inventors have developed techniques that address the above-described shortcomings associated with the conventional techniques for modeling genomic information. In some embodiments, the techniques include: (a) obtaining information specifying genomic context of the gene, the genomic context containing a plurality of genes including the gene, the information containing gene sequences for the plurality of genes; (b) encoding the information specifying the genomic context to obtain an initial encoding of the genomic context; and (c) processing the initial encoding of the genomic context with a genomic language model (gLM) to obtain the contextual embedding of the gene. In some embodiments, encoding the information specifying the genomic context includes: (a) mapping the gene sequences to protein sequences; and (b) encoding the protein sequences using a trained protein language model (pLM) to obtain the initial encoding of the genomic context. The resulting contextual embedding of the gene may be used in a wide variety of applications including, for example, identifying the function of a protein corresponding to the gene.

By accounting for multiple genes within a genomic context and representing those genes in continuous space, the techniques developed by the inventors can be used to generate contextual embeddings that capture complex relationships between the genes and their multi-dimensional properties. Thus, the generated contextual embeddings more accurately, comprehensibly, and reliably represent particular genes as they exist within their respective genomic contexts. Such an embedding can then be used in a variety of different applications. For example, the embedding may be used to identify a function of a protein and predict paralogy in protein-protein interactions, among other applications. Thus, generating a protein embedding in accordance with the techniques developed by the inventors and described herein is an improvement over conventional methods for generating protein embeddings (e.g., by using conventional protein language models). Thus, the techniques developed by the inventors provide an improvement to computational protein modeling technology, protein engineering technology, and machine learning technology for protein analysis, among other areas.

Following below are descriptions of various concepts related to, and embodiments of, techniques for generating a contextual embedding of a protein. It should be appreciated that various aspects described herein may be implemented in any of numerous ways, as the techniques are not limited in any particular manner of implementation. Example details of implementations are provided herein solely for illustrative purposes. Furthermore, the techniques disclosed herein may be used individually or in any suitable combination, as aspects of the technology described herein are not limited to the use of any particular technique or combination of techniques.

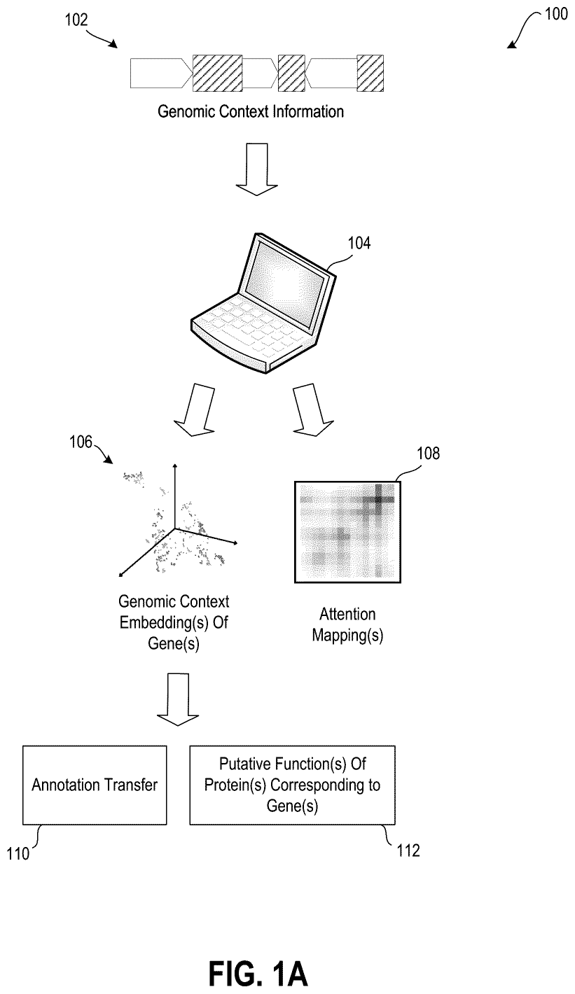

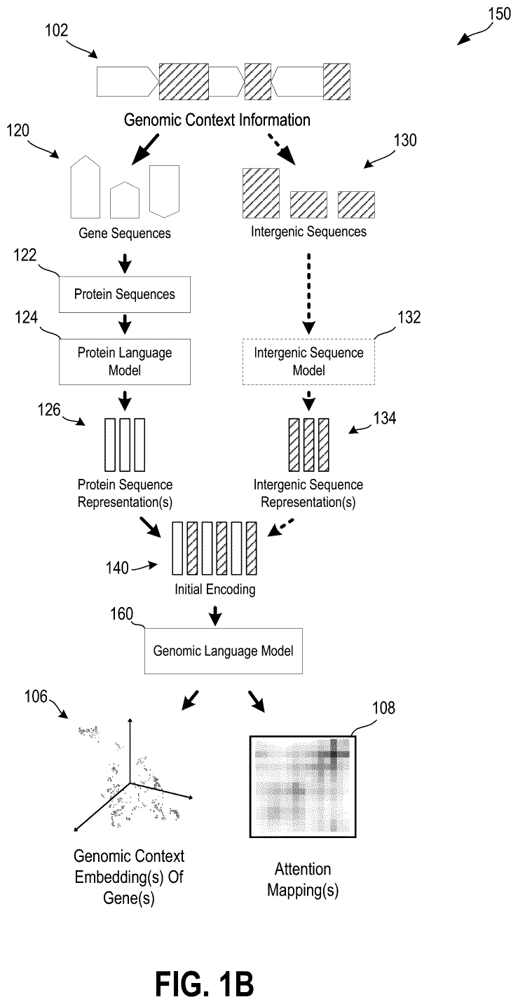

FIG. 1A is a diagram depicting an illustrative technique 100 for generating a contextual embedding of a gene, according to some embodiments of the technology described herein. Illustrative technique 100 includes processing genomic context information 102 for one or more genes using computing device 104 to predict genomic context embedding(s) 106 of the gene(s) and/or attention mapping(s) 108. The genomic context embedding(s) 106 may be used for one or more applications including, by way of example, for annotation transfer 110 and/or to identify putative function(s) 112 of protein(s) corresponding to the gene(s).

Genomic context information 102 may be obtained for one or more candidate gene(s). For example, a candidate gene may be a gene for which a genomic context embedding 106 is to be predicted. The one or more candidate genes may include any suitable number of genes such as a number of genes between 1 and 20,000, between 1 and 15,000, between 1 and 10,000, between 1 and 5,000, between 1 and 1,000, between 1 and 500, between 1 and 250, between 1 and 200, between 1 and 100, between 1 and 50, between 1 and 25, between 1 and 20, between 1 and 15, between 1 and 10, between 1 and 5, or a number of genes within any other suitable range, as aspects of the technology described herein are not limited in this respect. For example, illustrative technique 100 or parts thereof may be repeated for each of at least some (e.g., all) of the genes for which genomic context information 102 is obtained.

In some embodiments, the genomic context information 102 specifies the genomic context of a candidate gene. The genomic context may include a plurality of genes including the gene. For example, the genomic context may include a number of genes between 2 and 100, between 5 and 75, between 10 and 50, between 20 and 40, between 25 and 35, between 15 and 30, or a number of genes within any other suitable range, as aspects of the technology described herein are not limited in this respect. For example, the genomic context may include 30 genes including the candidate gene. In some embodiments, the genomic context may also include a plurality of intergenic regions. For example, the genomic context may include an intergenic region between at least some (e.g., all) pairs of adjacent genes of the plurality of genes included in the genomic context. In some embodiments, the genomic context is a gene subcontig containing the plurality of gene and/or intergenic regions. A subcontig may include a non-gapped DNA segment.

In some embodiments, the genomic context information 102 includes sequences for each of at least some (e.g., all) of the plurality of genes and/or intergenic regions included in the genomic context. For example, the genomic context information 102 may include gene sequences for (e.g., some or all of) the plurality of genes of the genomic context. The genomic context information 102 may also include a plurality of intergenic sequences for (e.g., some or all of) the intergenic regions included in the genomic context. In some embodiments, the gene sequences and intergenic sequences are sequences of nucleotides.

As shown in FIG. 1A, a computing device 104 may be used to process the genomic context information 102 to obtain the genomic context embedding(s) 106 and/or attention mapping(s) 108. In some embodiments, the computing device 104 may be operated by a user. For example, the user may provide genomic context information 102 as input to the computing device 104 (e.g., by uploading a file) and/or provide user input specifying processing or other methods to be performed on genomic context information 102. In some embodiments, computing device 104 may perform one or more calculations with respect to the sequence data without user intervention and, for example, can do so in response to receiving a request from a software program (e.g., via an API call) to do so. The computing device 104 may include one or more computing devices.

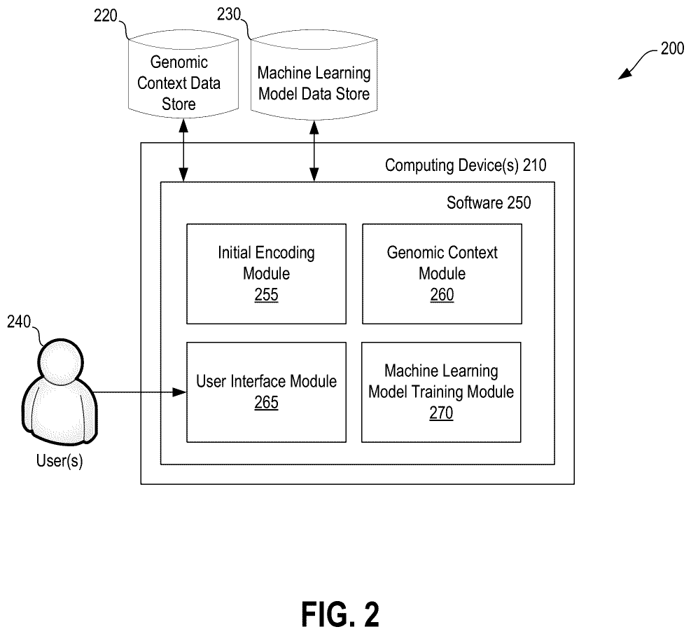

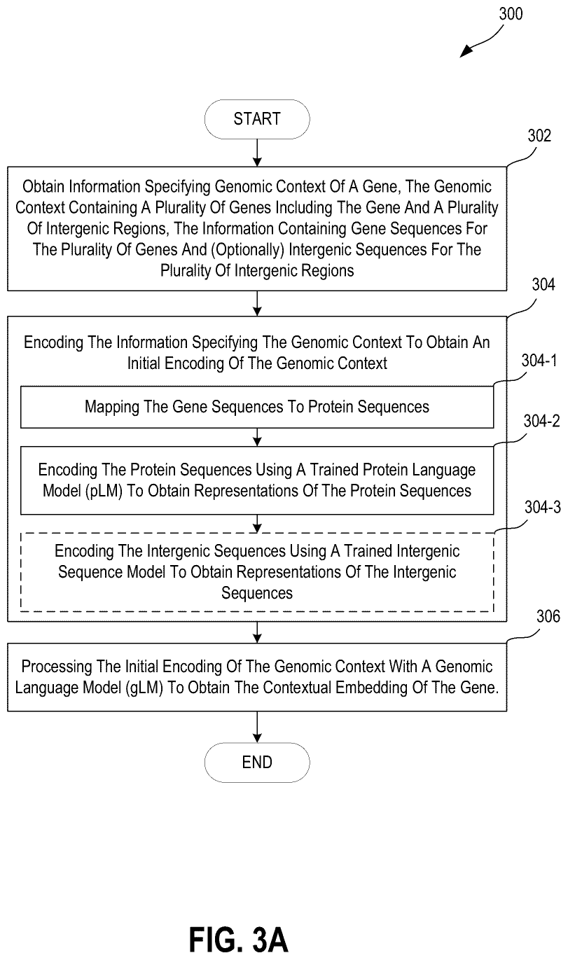

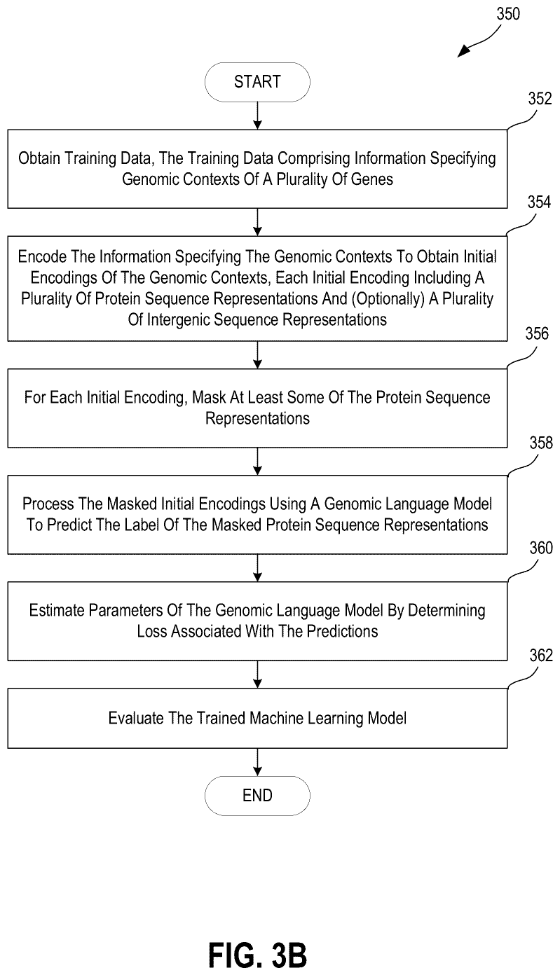

In some embodiments, software on the computing device 104 may be configured to process at least some (e.g., all) of the genomic context information 102 to obtain genomic context embedding(s) 106, attention mapping(s) 108, result(s) of annotation transfer 110, and/or putative functions(s) 112. In some embodiments, this may include: (a) encoding the genomic context information 102 to obtain an initial encoding of the genomic context, and (b) processing the initial encoding of the genomic context with a genomic language model (gLM) to obtain the contextual embedding 106 of the gene. In some embodiments, the contextual embedding 106 is used for annotation transfer 110 and/or to identify the putative function(s) 112 of a protein corresponding to the gene. Example techniques for processing genomic context information 102 using computing device 104 are described herein including at least with respect to FIG. 1B and process 300 shown in FIG. 3A. An example computing device 104 and such software are described herein including at least with respect to FIG. 2 (e.g., computing device(s) 210 and software 250).

In some embodiments, software on the computing device 104 may be configured to train a gLM to predict genomic context embedding(s) of gene(s). Example techniques for training a gLM are described herein including at least with respect to FIG. 3B. An example computing device 104 and such software are described herein including at least with respect to FIG. 2 (e.g., computing device(s) 210 and software 250).

As shown in FIG. 1A, the computing device 104 is configured to generate output(s) indicating genomic context embedding(s) 106, attention mapping(s) 108, annotation transfer result(s) 110, and/or putative function(s) 112 of protein(s) corresponding to the gene(s). In some embodiments, the output may be stored (e.g., in memory), displayed via a user interface, transmitted to one or more other devices, used to generate a report, and/or otherwise processed using any other suitable techniques, as aspects of the technology described herein are not limited in this respect. For example, the output of the computing device 104 may displayed using a graphical user interface (GUI) of a computing device (e.g., computing device 104).

In some embodiments, the genomic context embedding(s) 106 of gene(s) include the genomic context embedding(s) output by a trained gLM. In some embodiments, a genomic context embedding is an N-dimensional vector. In some embodiments, the value of N depends on the dimensional of one or more inputs to the gLM. In some embodiments, N is a value between 400 and 2400, between 800 and 1600, or a value within any other suitable range, as aspects of the technology described herein are not limited in this respect. For example, N may be 1280.

The genomic context embedding(s) 106 may be used for a variety of different applications. For example, the genomic context embedding(s) 106 may be used for annotation transfer 110. The annotation of a gene may include an indication of one or more functions of the gene. In some embodiments, annotation transfer 110 involves using the genomic context embedding 106 of a gene to annotate a previously unannotated gene. This may include comparing the genomic context embedding 106 of the unannotated gene to genomic context embedding(s) of annotated genes. Proteins found in similar genomic contexts, as captured by the genomic context embeddings, often confer similar functions due to the functional relationships between genes in a particular genomic context. Accordingly, in some embodiments, the annotation of an annotated gene may be used to annotate an unannotated gene when the genomic context embedding of the two genes are similar.

Additionally, or alternatively, the genomic context embedding(s) 106 of a particular gene may be used to identify one or more putative function(s) 112 of a protein corresponding to the particular gene. As described herein, understanding the functional role of a regulatory protein can be challenging because the same protein may carry out different functions in different contexts. Accordingly, the genomic context embedding(s) 106, which captures genomic context, can be used to predict the function of a protein corresponding to the particular gene. For example, a machine learning model may be trained (e.g., using feature-based transfer learning) to predict the function 112 of a protein corresponding to a particular gene given the genomic context embedding 106 of the gene.

As described herein, in some embodiments, an architecture of the gLM includes a plurality of layers. In some embodiments, a layer of the gLM includes one or more attention heads. In some embodiments, the attention mapping(s) 108 are self-attention weights for one or more of the attention heads. For example, as described herein, the self-attention weights may be extracted from an attention head after processing an initial encoding of the genomic context of a gene with the gLM. In some embodiments the attention mapping(s) 108 are two-dimensional (2D) arrays of self-attention weights. For example, an attention mapping for the gene may include an L×L array of self-attention weights, where L is the dimension of the initial representation of the gene of interest.

In some embodiments, attention mapping(s) 108 may be used to train a machine learning model to predict the presence of an operonic relationship between a pair of proteins encoded by neighboring genes within the genomic context 102. For example, the machine learning model may be a regression model (e.g., a logistic regression model). The machine learning model may be trained using attention mapping(s) 108 extracted from at least some (e.g., all) attention heads of the gLM.

FIG. 1B is a diagram depicting an illustrative technique 150 for processing information specifying genomic context of the gene to generate a contextual embedding 106 of a gene, according to some embodiments of the technology described herein. The illustrative technique 150 includes: (a) mapping gene sequences 120 to protein sequences 122, (b) processing the protein sequences using protein language model 124 to obtain initial encoding 140 which include includes protein sequence representation(s) 126, and (c) processing the initial encoding 140 using the genomic language model 160 to obtain genomic context embedding(s) 106 and/or attention mapping(s) 108. In some embodiments, illustrative technique 150 additionally includes processing intergenic sequences 130 using an intergenic sequence model 132 to obtain intergenic sequence representation(s) 134. In such embodiments, the initial encoding(s) 140 may include both protein sequence representation(s) 126 and intergenic representation(s) 134.

In some embodiments, gene sequences 120 are mapped to protein sequences 122. In some embodiments, mapping the gene sequences 120 to protein sequences 122 includes determining a sequence of amino acids that corresponds to the gene sequence. A sequence of amino acids that corresponds to a gene sequence may include the sequence of amino acids that may result from transcription of the gene sequence (e.g., a sequence of nucleotides). In some embodiments, the mapping is performed using software on the computing device 104. For example, the software MMseq2 and/or Linclust may be used to map the gene sequence to the protein sequence. MMseq2 is described by Steinegger, M. & Söding, J. (“MMseqs2 enables sensitive protein sequence searching for the analysis of massive data sets.” Nat. Biotechnol. 35, 1026-1028 (2017)) and is incorporated by reference herein in its entirety. Linclust is described by Steinegger, M. & Söding, J. (“Clustering huge protein sequence sets in linear time.” Nature communications 9.1 (2018): 2542) and is incorporated by reference herein in its entirety. However, any other suitable software may be used to perform the protein sequence mapping, as aspects of the technology described herein are not limited to a particular protein sequence mapping software. Additionally, or alternatively, the mapping may be obtained according to any other suitable techniques. For example, a user may specify the protein sequence(s) 122 and/or the computing device may otherwise obtain protein sequence(s) 122 that were previously determined.

In some embodiments, the protein language model 124 is used to process the protein sequences 122. The protein sequences 122 and/or representations of the protein sequences 122 may be provided as input to the protein language model 124. For example, a protein sequence may be represented using amino acid alphabets, encoded as one-hot representations. The protein language model may be any suitable protein language model trained to encode amino acid sequences by processing information representing an amino acid sequence to obtain a numeric output (e.g., a vector of real numbers) representing the encoding of the amino acid sequence (e.g., protein sequence representation(s) 126), as aspects of the technology described herein are not limited in this respect. Examples of protein language models include the ESM-1b model, the ESM-1v model, and the ESM-2 model. The ESM-1b model is described by Rives, A., et al. (“Biological structure and function emerge from scaling unsupervised learning to 250 million protein sequences.” Proceedings of the National Academy of Sciences 118.15 (2021): e2016239118.), which is incorporated by reference herein in its entirety. The ESM-1v model is described by Meier, J., et al. (“Language models enable zero-shot prediction of the effects of mutations on protein function.” Advances in Neural Information Processing Systems 34 (2021): 29287-29303.), which is incorporated by reference herein in its entirety. The ESM-2 model is described by Lin, Z., et al. (“Evolutionary-scale prediction of atomic-level protein structure with a language model.” Science 379.6637 (2023): 1123-1130.), which is incorporated by reference herein in its entirety.

Protein sequence representation(s) 126 are output by protein language model 124. As described herein, the protein sequence representation(s) 126 may be include numeric outputs (e.g., a vector of real numbers) representing the encoding of the protein sequences 122. For example, a protein sequence representation may include an N-dimensional vector. In some embodiments, N is a value between 400 and 2400, between 800 and 1600, or a value within any other suitable range, as aspects of the technology described herein are not limited in this respect. For example, N may be 1280.

In some embodiments, intergenic sequence model 132 is used to process intergenic sequences 130. The intergenic sequences 130 and/or representations of the intergenic sequences 130 may be provided as input to the intergenic sequence model 132. For example, an intergenic sequence may be represented using nucleotides (e.g., A, T, C, G), encoded as one-hot representations. In some embodiments, the intergenic sequence model 132 is a transformer model trained to predict a representation of an input intergenic sequence. The transformer model may include a plurality of layers. For example, the transformer model may include a number of layers between 2 and 20, between 3 and 18, between 4 and 16, between 5 and 15, between 6 and 14, between 7 and 13, between 8 and 12, between 9 and 11, or between any other suitable range, as aspects the technology described herein are not limited in this respect. A layer of the transformer model may have any suitable dimensionality. For example, the dimensionality of a layer may be a dimensionality between 250 and 1,000, between 300 and 800, between 400 and 600, or a dimensionality within any other suitable range, as aspects of the technology described herein are not limited in this respect. For example, the transformer model may have 10 layers of dimensionality 512. In some embodiments, each of one or more of the layers includes one or more attention heads. For example, a layer may include between 2 and 20, between 3 and 15, between 4 and 12, between 5 and 10, between 6 and 9, or between any other suitable range, as aspects of the technology described herein are not limited in this respect. For example, the transformer model may have 8 attention heads.

Intergenic sequence representation(s) 134 are output by intergenic sequence model 132. As described herein, the intergenic sequence representation(s) 134 may be include numeric outputs (e.g., a vector of real numbers) representing the encoding of the intergenic sequences 130. For example, an intergenic sequence representation may include an N-dimensional vector. In some embodiments, N is a value between 400 and 2400, between 800 and 1600, or a value within any other suitable range, as aspects of the technology described herein are not limited in this respect. For example, N may be 1280.

Initial encoding(s) 140 may include protein sequence representation(s) 126 and (optionally) intergenic sequence representation(s) 134. For example, initial encoding 140 may include an K×N array, where K represents the number of protein sequence representations or intergenic sequence representations, and N represents the number of features in each representation. For example, where each protein and/or intergenic sequence representation is an 1280-dimensional vector and there is a total of 30 genes and/or intergenic sequences, the array have a size of 1280×30. The order of the protein and/or intergenic sequence representations within the array may depend on the order in which the corresponding genes and intergenic regions appear in the genomic context, as indicated by information 102.

The genomic language model 160 is used to process the initial encoding 140 to obtain the genomic context embedding(s) 106 and/or attention mapping(s) 108. In some embodiments, the genomic language model 160 is a transformer model (e.g., a multi-layer transformer model). The transformer model may including a number of layers between 5 and 50, 10 and 45, 20 and 40, or a number of layers within any other suitable range, as aspects of the technology described herein are not limited in this respect. For example, the transformer model may include 36 layers. The transformer model may include multiple hidden layers including a number of hidden layers between 5 and 35, 10 and 25, 15 and 20, or a number of hidden layers within any other suitable range, as aspects of the technology described herein are not limited in this respect. For example, the transformer model may include 19 hidden layers. In some embodiments, at least one (e.g., each) hidden layer includes one or more attention heads. For example, a hidden layer may include between 5 and 15 attention heads, or a number of attention heads within any other suitable range, as aspects of the technology described herein are not limited in this respect. For example, a hidden layer may include 10 attention heads. In some embodiments, the architecture of the transformer model is built on an implementation of the ROBERTa transformer architecture, or any other suitable transformer architecture. RoBERTa is described by Liu, Y. et al. (“RoBERTa: A Robustly Optimized BERT Pretraining Approach.” arXiv [cs.CL] (2019).), which is incorporated by reference herein in its entirety. Example techniques for training a genomic language model are described herein including at least with respect to FIG. 3B.

In some embodiments, the genomic context embedding 106 of a gene is obtained from one or more hidden states of the genomic language model 160. For example, the genomic context embedding 106 may be obtained from the last hidden state of the genomic language model. In some embodiments, the genomic context embedding is obtained by mean-pooling the last hidden state. The genomic context embedding may be a numeric representation (e.g., an M-dimensional vector) of the gene, where M may be equal to the dimension N of the input provided to the gLM.

FIG. 2 is a block diagram of an example system 200 for generating a contextual embedding of a gene, according to some embodiments of the technology described herein. System 200 includes computing device(s) 210 configured to have software 250 execute thereon to perform various functions in connection with training and using a genomic language model (gLM) to generate a contextual embedding of a gene. In some embodiments, software 250 includes a plurality of modules. A module may include processor-executable instructions that, when executed by at least one computer hardware processor, cause the at least one computer hardware processor to perform function(s) of the module. Such modules are sometimes referred to herein as “software modules,” each of which includes processor-executable instructions configured to perform one or more acts of one or more processes, such as process 300 shown in FIG. 3A and process 350 shown in FIG. 3B.

The computing device(s) 210 may be operated by one or more user(s) 240. In some embodiments, the user(s) 240 may provide, as input to the computing device(s) 210 (e.g., by uploading one or more files, by interacting with a user interface of the computing device(s) 210, etc.) information specifying genomic context data of a gene (e.g., sequence data). Additionally, or alternatively, the user(s) 240 may provide input specifying processing or other methods to be performed on the information specifying the genomic context data of the gene. Additionally, or alternatively, the user(s) 240 may access results of processing the information specifying the genomic context data of the gene. For example, the user(s) 240 may access a contextual embedding of the gene, one of more putative functions of a protein corresponding to the gene, one or more attention mappings from the gLM, or any other suitable results, as aspects of the technology described herein are not limited in this respect.

In some embodiments, the initial encoding module 255 obtains information specifying the genomic context of a gene. For example, the initial encoding module 255 may obtain the information from the genomic context data store 220 and/or user(s) 240. The information may include sequences for a plurality of genes (e.g., including the gene of interest) and/or sequences for a plurality of intergenic regions.

In some embodiments, the initial encoding module 255 obtains one or more trained machine learning models. For example, the initial encoding module 255 may obtain the one or more trained machine learning models from the machine learning model data store 230 and/or machine learning model training module 270. The one or more trained machine learning models may include, for example, a trained protein language model and/or a trained transformer model.

In some embodiments, the initial encoding module 255 is configured to obtain an initial encoding of the genomic context of a gene. To this end, in some embodiments, the initial encoding module 255 is configured to: (a) map gene sequence(s) to protein sequence(s), (b) encode the protein sequence(s) to obtain representation(s) of the protein sequence(s), and/or (c) encode intergenic sequence(s) to obtain representation(s) of the intergenic sequence(s).

In some embodiments, the initial encoding module 255 is configured to map gene sequence(s) to protein sequence(s). For example, the initial encoding module 255 may be configured to determine the sequence amino acids that may result from transcription of the gene sequence (e.g., a sequence of nucleotides). In some embodiments, the initial encoding module 255 is configured to use protein mapping software to map the gene sequence(s) to the protein sequence(s). For example, the initial encoding module 255 may use MMseqs2, which is described by Steinegger, M. & Söding, J. (“MMseqs2 enables sensitive protein sequence searching for the analysis of massive data sets.” Nat. Biotechnol. 35, 1026-1028 (2017)) and is incorporated by reference herein in its entirety. Additionally, or alternatively, the initial encoding module 255 may use Linclust, which is described by Steinegger, M. & Söding, J. (“Clustering huge protein sequence sets in linear time.” Nature communications 9.1 (2018): 2542) and is incorporated by reference herein in its entirety. Example techniques for mapping gene sequence(s) to protein sequence(s) are described herein including at least with respect to FIG. 1B and act 304-1 of process 300 shown in FIG. 3A.

In some embodiments, the initial encoding module 255 is configured to encode one or more protein sequence(s) to obtain representation(s) of the protein sequence(s). The initial encoding module 255 may be configured to obtain a trained protein language model (e.g., from machine learning model data store 230) and encode the protein sequence(s) using the trained protein language model. For example, the initial encoding module 255 may be configured to process the protein sequence(s) using the ESM-2 protein language model, the ESM-1b language model, and/or the ESM-1v protein language model to obtain numeric representation(s) of the protein sequence(s). Example techniques for encoding a protein sequence using a trained protein language model are described herein including at least with respect to FIG. 1B and act 304-2 of process 300 shown in FIG. 3.

In some embodiments, the initial encoding module 255 is configured to encode one or more intergenic sequence(s) to obtain representation(s) of the intergenic sequence(s). The initial encoding module 255 may be configured to obtain a trained intergenic sequence model (e.g., from machine learning model data store 230) and encode the intergenic sequence(s) using the trained intergenic sequence model. Examples of an intergenic sequence model trained to encode intergenic sequence(s) are described herein including at least with respect to FIG. 1B and act 304-3 of process 300 shown in FIG. 3A.

In some embodiments, the genomic context module 260 obtains an initial encoding of the genomic context of a gene. For example, the genomic context module 260 may obtain initial encoding(s) from the initial encoding module 255, user(s) 240, and/or genomic context data store 220. The initial encoding(s) may include one or more representation(s) of the protein sequence(s) and/or one or more representation(s) intergenic sequence(s).

In some embodiments, the genomic context module 260 obtains one or more trained machine learning models. For example, the genomic context module 260 may obtain one or more trained machine learning model(s) from the machine learning model data store 230 and/or machine learning model training module 270. The one or more trained machine learning models may include, for example, a trained genomic language model (gLM).

In some embodiments, the genomic context module 260 is configured to process an initial encoding of the genomic context of a gene using a gLM. In some embodiments, the genomic context module 260 is configured to obtain a trained gLM (e.g., from the machine learning model data store 230) and process the initial encoding using the obtained gLM. For example, the genomic context module 260 may process the initial encoding using the obtained gLM to obtain a contextual embedding of the gene. Additionally, or alternatively, the genomic context module 260 may process the initial encoding using the obtained gLM to obtain one or more attention mappings. Examples of training and using a gLM are described herein including at least with respect to FIG. 1B, act 306 of process 300 shown in FIG. 3A, and process 350 shown in FIG. 3B.

In some embodiments, the genomic context module 260 is configured to identify a putative function of a protein corresponding to the gene. For example, the genomic context module 260 may be configured to obtain, for one or more other genes, information indicating: (i) contextual embedding of the gene(s), and (ii) function(s) associated with the gene(s). For example, the genomic context module 260 may obtain the information for the other gene(s) from the genomic context data store 220 and/or user(s) 240. To determine a putative function of the protein corresponding to the gene, the genomic context module may be configured to compare the contextual embedding obtained for the gene to the contextual embedding(s) for the other gene(s).

In some embodiments, the machine learning model training module 270 is configured to train one or more machine learning models to encode one or more intergenic sequences. For example, the machine learning model training module 270 may obtain training data (e.g., intergenic sequence(s)) from the genomic context data store 220 and/or user(s) 240 (e.g., by the user(s) 240 uploading the training data). The machine learning model training module 270 may be configured to use the obtained training data to train a intergenic sequence model to encode one or more intergenic sequences. In some embodiments, the machine learning model training module 270 may provide the trained intergenic sequence model to the machine learning model data store 230 for storage thereon. For example, the machine learning model training module 270 may provide the values of parameters of the intergenic sequence model to the machine learning model data store 230 for storage thereon.

In some embodiments, the machine learning model training module 270 is configured to train one or more machine learning models to predict the contextual embedding of a gene. For example, the machine learning model training module 270 may obtain training data (e.g., initial encoding(s) of the genomic context(s)) from the initial encoding module 255, genomic context data store 220, and/or user(s) 240 (e.g., by the user(s) 240 uploading the training data). The machine learning model training module 270 may be configured to use the obtained training data to train a gLM to predict contextual embedding(s) of gene(s). In some embodiments, the machine learning model training module 270 may provide the trained gLM to the machine learning model data store 230 for storage thereon. For example, the machine learning model training module 270 may provide the values of parameters of the gLM to the machine learning model data store 230 for storage thereon. Techniques for training a gLM to predict a contextual embedding of a gene are described herein including at least with respect to FIG. 3B.

In some embodiments, the genomic context data store 220 stores training data used to train one or more machine learning models. For example, the training data may include training data for training a intergenic sequence model to encode intergenic sequence(s). Additionally, or alternatively, the training data may include training data for training a gLM to predict a contextual embedding of a gene. The training data may include information specifying the genomic context of a plurality of genes. For example, the training data may include a plurality of gene sequences and/or representations thereof. Additionally, or alternatively, the training data may include a plurality of intergenic sequences and/or representations thereof.

In some embodiments, the genomic context data store 220 stores information specifying the genomic context of one or more candidate genes. For example, the one or more candidate genes may include genes for which a contextual embedding is to be obtained. The information specifying the genomic context of a candidate gene may include a plurality of gene sequences and/or representations thereof. Additionally, or alternatively, the training data may include a plurality of intergenic sequences and/or representations thereof.

The genomic context data store 220 includes any suitable type of data store (e.g., a flat file, a database system, a multi-file, etc.) and may store data in any suitable format, as aspects of the technology described herein are not limited in this respect. The genomic context data store 220 may be part of software 250 (not shown) or excluded from software 250, as shown in FIG. 2.

In some embodiments, the machine learning model data store 230 stores one or more machine learning models. For example, the machine learning model data store 230 may store a gLM trained to predict a contextual embedding of a gene. Additionally, or alternatively, the machine learning model data store 230 may store one or more protein language models trained to encode protein sequences. Additionally, or alternatively, the machine learning model data store 230 may store one or more intergenic sequence models trained to encode intergenic sequences. In some embodiments, the machine learning model data store 230 includes any suitable type of data store such as a flat file, a database system, a multi-file, or data store of any suitable type, as aspects of the technology described herein are not limited to any particular type of data store. The machine learning model data store 230 may be part of software 250 (not shown) or excluded from software 250, as shown in FIG. 2. In some embodiments, the machine learning model data store 230 stores parameter values for trained machine learning model(s). When the stored trained machine learning model(s) are loaded and used, for example by initial encoding module 255 and/or genomic context module 260, the parameter values of the trained machine learning model are loaded and stored in memory using at least one data structure.

As shown in FIG. 2, software 250 also includes user interface module 265. User interface module 265 may be configured to generate a graphical user interface (GUI) through which user(s) 240 may provide input and view information generated by software 250. For example, in some embodiments, the user interface module 265 may be a webpage or web application accessible through an Internet browser. In some embodiments, the user interface module 265 may generate a GUI of an app executing on a user's mobile device. In some embodiments, the user interface module 265 may generate a number of selectable elements through which a user may interact. For example, the user interface module 265 may generate dropdown lists, checkboxes, text fields, or any other suitable element.

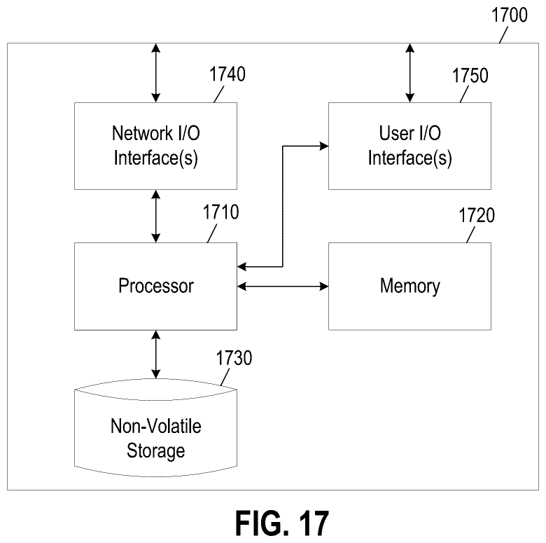

FIG. 3A is a flowchart of an illustrative process 300 for generating a contextual embedding of a gene, according to some embodiments of the technology described herein. One or more (e.g., all) of the acts of process 300 may be performed automatically by any suitable computing device(s). For example, the act(s) may be performed by a laptop computer, a desktop computer, one or more servers, in a cloud computing environment, computing device(s) 210 as described herein including at least with respect to FIG. 2, computing system 1700 as described herein including at least with respect to FIG. 17, and/or in any other suitable way, as aspects of the technology described herein are not limited in this respect.

At act 302, information is obtained specifying the genomic context of a gene. The genomic context includes a plurality of genes and intergenic regions. The plurality of genes includes the gene for which a contextual embedding is to be obtained. In some embodiments, the obtained information includes gene sequences for genes contained in the genomic context and, optionally, intergenic sequences for the intergenic regions contained in the genomic context. Examples of information specifying the genomic context of a gene and techniques for obtaining same are described herein including at least with respect to FIGS. 1A-1B and 2.

At act 304, the information specifying the genomic context is encoded to obtain an initial encoding of the genomic context. In some embodiments, the initial encoding includes representations of protein sequences corresponding to the gene sequences obtained at act 302. Additionally, the initial encoding may include representations of the intergenic sequences obtained at act 302. Examples of an initial encoding of a genomic context are described herein including at least respect to initial encoding 140 shown in FIG. 1B. In some embodiments, encoding the information to obtain the initial encoding includes performing one or more of acts 304-1, 304-2, and 304-3.

At act 304-1, the gene sequences are mapped to protein sequences. Example techniques for mapping gene sequences to protein sequences are described herein including at least with respect to FIG. 1B and FIG. 2.

At act 304-2, the protein sequences are encoded using a trained protein language model to obtain representations of the protein sequences. Example techniques for encoding protein sequences using trained protein language model are described herein including at least with respect to FIG. 1B and FIG. 2.

At (optional) act 304-3 the intergenic sequences are encoded using a trained intergenic sequence model to obtain representations of the intergenic sequences. Example techniques for encoding intergenic sequences using an intergenic sequence model are described herein including at least with respect to FIG. 1B and FIG. 2.

At act 306, the initial encoding of the genomic context is processed using a genomic language model (gLM) to obtain the contextual embedding of the gene. Example techniques for processing an initial encoding of a genomic context using a gLM are described herein including at least with respect to FIG. 1B and FIG. 2.

FIG. 3B is a flowchart of an illustrative process 350 for training a genomic language model (gLM) to obtain a contextual embedding of a gene, according to some embodiments of the technology described herein. One or more (e.g., all) of the acts of process 300 may be performed automatically by any suitable computing device(s). For example, the act(s) may be performed by a laptop computer, a desktop computer, one or more servers, in a cloud computing environment, computing device(s) 210 as described herein including at least with respect to FIG. 2, computing system 1700 as described herein including at least with respect to FIG. 17, and/or in any other suitable way, as aspects of the technology described herein are not limited in this respect.

At act 352, training data is obtained. In some embodiments, the training data includes information specifying the genomic context of each of a plurality of genes. Information specifying genomic context of a gene is described herein including at least with respect to FIG. 1A and FIG. 1B (e.g., genomic context information 102). The training data may include information for any suitable number of genomic contexts including a number of genomic contexts between 500,000 and 15,000,000, between 1,000,000 and 12,000,000, between 3,000,000 and 10,000,000, between 5,000,000 and 8,000,000, or a number of genomic contexts within any other suitable range, as aspects of the technology described herein are not limited in this respect. The information for a particular genomic context may include one or more gene sequences and (optionally) one or more intergenic sequences.

At act 354, the genomic context information is encoded to obtain initial encodings of the genomic contexts of the genes. In some embodiments, this includes encoding the information specifying each of at least some (e.g., all) of the genomic contexts for which training data was obtained at act 352. As described herein, encoding the information specifying the genomic context of a gene may include: (a) mapping gene sequences to protein sequences, (b) encoding the protein sequences using a trained protein language model to obtain representations of the protein sequences, and (c) (optionally) encoding the intergenic sequences using a trained intergenic sequence model to obtain representations of the intergenic sequences. Example techniques for encoding the information specifying the genomic context of a gene are described herein including at least with respect to act 304 of process 300, FIG. 1B, and FIG. 2. Additionally, or alternatively, the initial encodings may be obtained with training data at act 302. For example, the encoding may have been performed prior to process 350.

In some embodiments, a gene orientation feature is added to each of at least some (e.g., all) protein sequence representations in an initial encoding. For example, the gene orientation feature may provide a binary indication as to whether the corresponding gene is in a “forward” or “reverse” orientation relative to the direction of sequencing. For example, 0.5 may denote the forward orientation and-0.5 may denote the reverse direction. Accordingly, this may increase the size of the numeric representation (e.g., N-dimensional vector) of a protein sequence. For example, a 1280-dimensional vector representing a protein sequence may increase to a 1281-dimensional vector with the addition of the gene orientation feature.

At act 356, for each initial encoding, at least some of the protein sequence representations are masked. In some embodiments, this includes masking a particular number or percentage of protein sequence representations in a particular initial encoding. For example, this may include masking between 5% and 25% of the protein sequence representations contained in the initial encoding. For example, 15% of the protein sequence representations may be masked. In some embodiments, the protein sequence representations are randomly masked. In some embodiments, masking a protein sequence representation includes masking the protein sequence representation to a particular value (e.g., −1).

At act 358, the masked initial encodings are processed using the genomic language model to predict labels of the masked protein sequence representations. In some embodiments, the label includes a reduced-dimensionality feature vector. For example, the label may be a 100-feature vector that include principal component analysis (PCA) whitened 99 principal components. In some embodiments, the genomic language model projects a hidden state (e.g., the last hidden state) onto one or more feature vectors (e.g., at least 1, at least 2, at least 3, at least 4, etc.) and corresponding likelihood values using a linear layer.

At act 360, estimate parameters of the genomic language model by determining loss associated with predictions. In some embodiments, the parameters are estimated by applying a loss function to the label and the prediction (e.g., the feature vector) closest to the label. In some embodiments, the prediction closest to the label is determined based on L2 distance. In some embodiments, the loss is calculated using Equation 1:

MSE(closest prediction, label)+α*CrossEntropyLoss(likelihoods, closest prediction index) (Equation 1)

where the learning rate α is any suitable learning rate set using any suitable techniques. For example, α may be le-4. In some embodiments, an optimizer is used to adjust parameters and the learning rate to reduce loss. For example, the AdamW optimizer may be used. AdamW is described by Loshchilov, I. & Hutter, F. (“Decoupled Weight Decay Regularization.” arXiv [cs.LG] (2017).), which is incorporated by reference herein in its entirety.

At act 362, the trained genomic language model is evaluated. In some embodiments, the trained genomic language model is evaluated by determining a pseudo-accuracy metric. The pseudo accuracy metric may deem a prediction to be correct if it is the closest in Euclidian distance to the label of the masked protein sequence representation relative of other protein sequence representations in the genomic context. Pseudo-accuracy may be calculated using Equation 2:

$$pseudo accuracy = \frac{\begin{matrix}\# count ( argmin ( dist ( prediction , labels in \\ subcontig ) ) == index ( masked gene ) )\end{matrix}}{( \# masked genes )} \qquad ( Equation 2 )$$

### EXAMPLES

This example relates to a genomic language model (gLM) that was developed to learn the contextual representations of genes. gLM leverages pLM embeddings as input, which encode relational properties and structure information of the gene products. This model is based on the transformer architecture and is trained using millions of unlabelled metagenomic sequences via the masked language modeling objective, with the hypothesis that its ability to attend to different parts of a multi-gene sequence will result in the learning of gene functional semantics and regulatory syntax (e.g. operons). Presented herein is evidence of the learned contextualized protein embeddings and attention patterns capturing biologically relevant information. gLM's potential for predicting gene function and co-regulation is demonstrated herein. This example includes the following sections: “Results” and “Methods.”

### Results

### Masked Language Modeling of Genomic Sequences

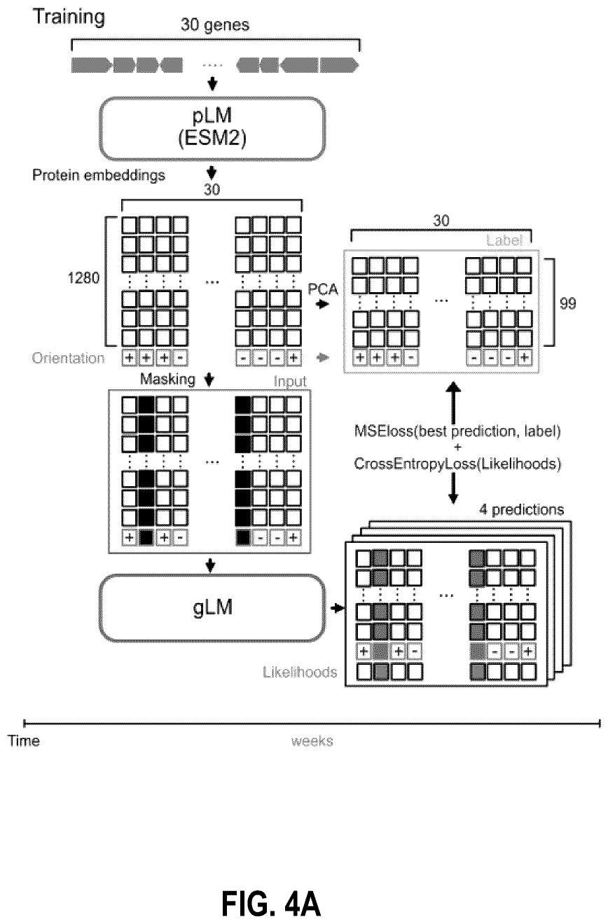

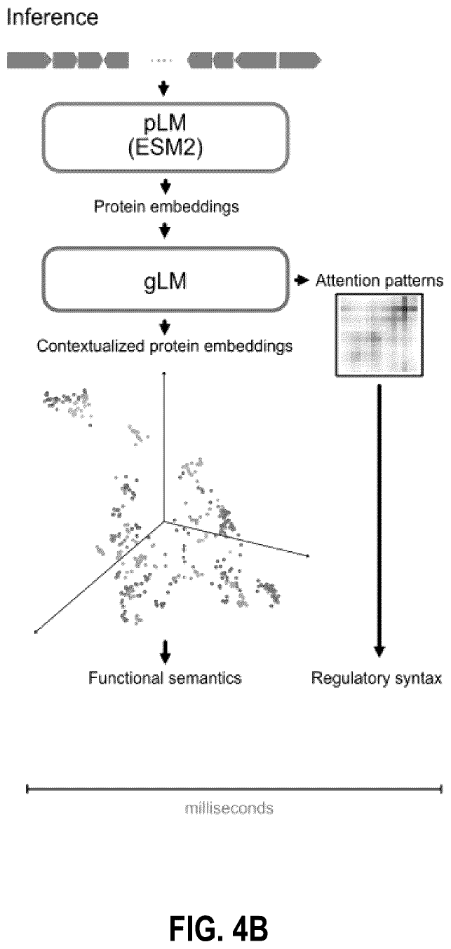

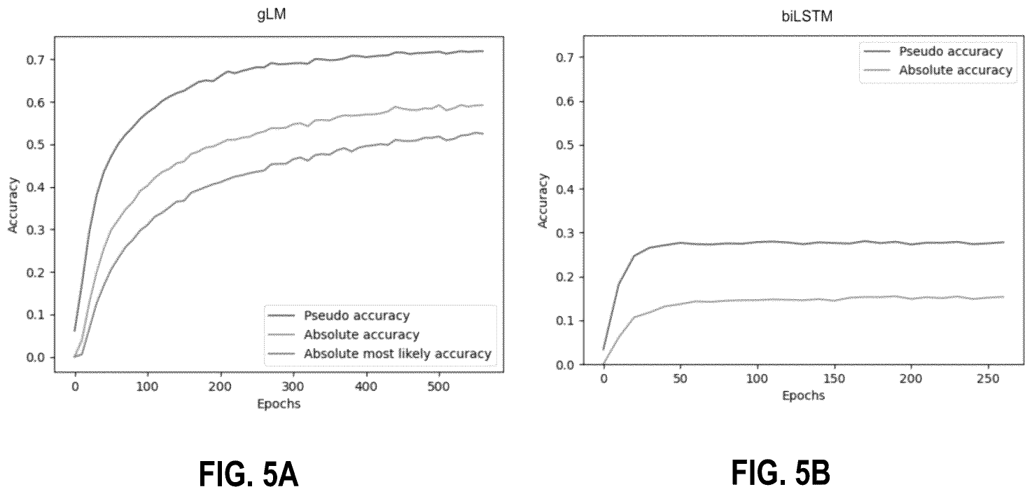

Language models, such as Bidirectional Encoder Representations from Transformers (BERT), learn the semantics and syntax of natural languages using unsupervised training of a large corpus. In masked language modeling, the model is tasked with reconstructing corrupted input text, where some fraction of the words are masked. Significant advances in language modeling performance was achieved by adopting the transformer neural network architecture, where each token (i.e. word) is able to attend to other tokens. This is in contrast to Long-Short-Term-Memory networks (LSTMs) that sequentially processes tokens. To model genomic sequences, a 19-layer transformer model (FIGS. 4A-4B) was trained on seven million metagenomic contig fragments consisting of 15 to 30 genes from the MGnify database. Each gene in a genomic sequence is represented by a 1280 feature vector (context-free protein embeddings) generated by using ESM2 pLM, concatenated with an orientation feature (forward or backward). For each sequence, 15% of genes are randomly masked, and the model learns to predict the masked label using the genomic context. Based on the insight that more than one gene can legitimately be found in a particular genomic context, the model was allowed to make four different predictions and also predict their associated probabilities (FIGS. 4A-4B). Thus, instead of predicting their mean value, the model can approximate the underlying distribution of multiple genes that can occupy a genomic niche. The model's performance was assessed using a pseudo-accuracy metric, where a prediction is considered correct if it is closest to the masked protein in euclidean distance compared to the other proteins encoded in the sequence (see “Methods”). The model's performance was validated on the Escherichia coli K-12 genome by excluding from training 5.1% of MGnify subcontigs in which more than half of the proteins are similar (>70% sequence identity) to E. coli K-12 proteins. The goal was not to remove all E. coli K-12 homologs from the training, which would have removed a vast majority of training data as many essential genes are shared across organisms. Instead, the goal was to remove as many E. coli K-12-like genomic contexts (subcontigs) from training, which is more appropriate for the training objective. gLM achieves 71.9% in validation pseudo-accuracy and 59.2% in validation absolute accuracy (FIGS. 5A-5B). Notably, 53.0% of the predictions made during validation are with high confidence (with prediction likelihood >0.75), and 75.8% of the high confidence predictions are correct, indicating gLM's ability to learn a confidence metric that corresponds to increased accuracy. The performance was baselined with a bidirectional LSTM model trained using the same language modeling task on the same training dataset, where validation performance plateaus at 28% pseudo-accuracy and 15% absolute accuracy (FIGS. 5A-5B and Table 2, note that biLSTM is smaller because it failed to converge when scaling the number of layers).

TABLE 2
Comparison of biLSTM baseline model with transformer-based
gLM architecture and validation performances. Note that
the biLSTM baseline is smaller in model size than gLM.
While scaling this model was attempted by increasing the
number of layers, the model failed to converge.

| | biLSTM | gLM |
|---|---|---|
| Number of layers | 5 | 19 |
| Attention heads | N/A | 10 |
| Input embedding dimension | 1281 | 1281 |
| Hidden size | 1280 | 1280 |
| Batch size | 4000 | 3000 |
| Learning rate | 1e−4 | 1e−4 |
| Warm up steps | 5000 | 5000 |
| Training steps | 467,253 | 1,296,960 |
| Number of predictions | 1 | 4 |
| Number of parameters | 27,811,840 | 954,736,916 |
| % Pseudo-accuracy (validation) | 27.9 | 71.9 |
| % Absolute accuracy (validation) | 14.78 | 59.2 |

pLM representations were replaced, as input to gLM, with one-hot amino acid representations (Table 3). Performance equivalent to random predictions (3% pseudo-accuracy and 0.02% absolute accuracy) is reported.

TABLE 3
Ablation of pLM representations. Ablated gLM was
trained on one-hot representations until convergence
(<0.1% decrease in loss over 40k iterations).

| | gLM | gLM one-hot |
|---|---|---|
| Representations | ESM2 embedding | One-hot amino |
| | | acid encoding |
| Pooling | mean | mean |
| Number of layers | 19 | 19 |
| Attention heads | 10 | 10 |
| Input embedding dimension | 1281 | 34 |
| Hidden size | 1280 | 1280 |
| Batch size | 3000 | 3000 |
| Learning rate | 1e−4 | 1e−4 |
| Warm up steps | 5000 | 5000 |
| Training steps | 1296960 | 132050 |
| Number of predictions | 4 | 4 |
| Number of parameters | 954736916 | 945338764 |
| % Pseudo-accuracy (validation) | 71.9 | 3.29 |
| % Absolute accuracy (validation) | 59.2 | 0.002 |
| Operon prediction mAP | 0.775 ± 0.29 | 0.426 ± 0.015 |

Contextualized gene Embeddings Capture gene Semantics

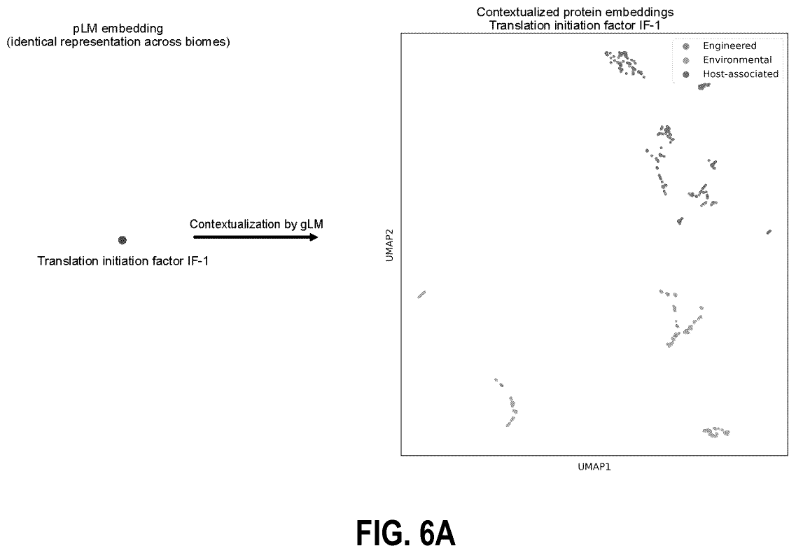

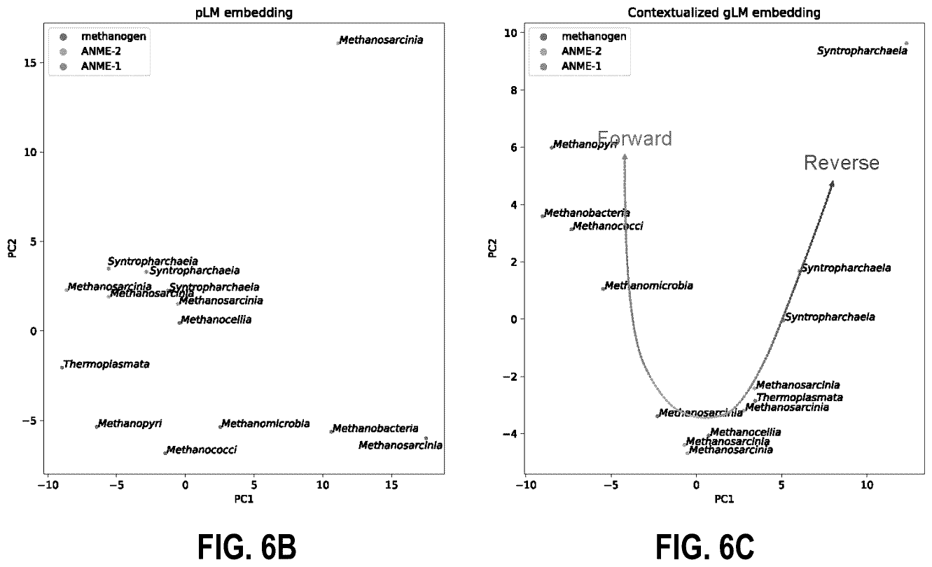

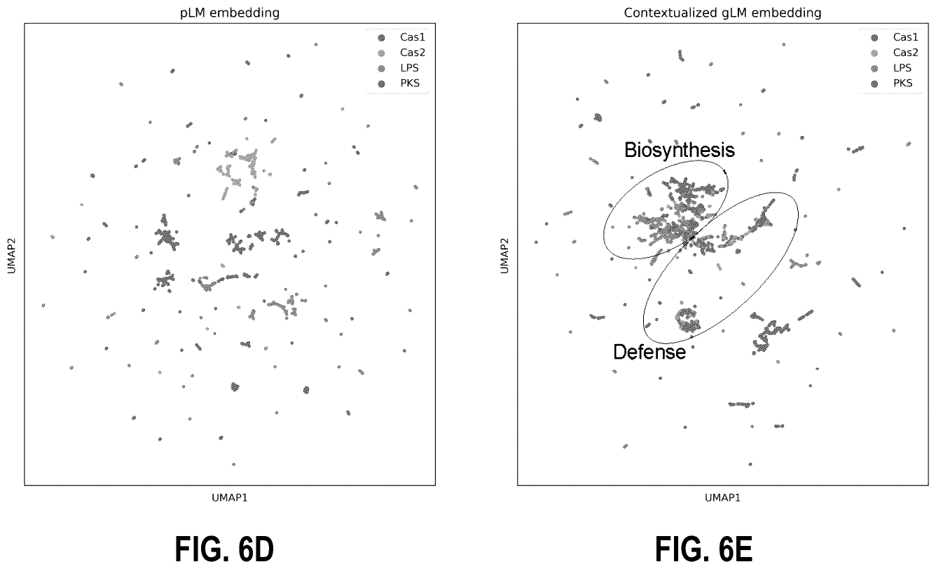

The mapping from gene to gene-function in organisms is not one-to-one. Similar to words in natural language, a gene can confer different functions depending on its context, and many genes confer similar functions (i.e. convergent evolution, remote homology). gLM was used to generate 1280-feature contextualized protein embeddings at inference time (FIG. 4B), and the “semantic” information captured in these embeddings was examined. Analogous to how words are likely to have different meanings depending on the type of text in which they are found, it was found that contextualized protein embeddings of genes that appear across multiple environments (biomes) tend to cluster based on biome types. 31 proteins were identified in the training database (MGYPs) that occurred more than 100 times and distributed with at least 20 occurrences in each “Host-associated”, “Environmental”, and “Engineered” biomes according to MGnify's designation. It was found that gLM's contextualized protein embeddings capture biome information for the majority (n=21) of these multi-biome MGYPs. For instance, a gene encoding a protein annotated “translation initiation factor IF-1” occurs multiple times across biomes. While the input to gLM (context-free protein embedding; ESM2 representation) is identical across all occurrences, gLM's output (contextualized protein embeddings) cluster with biome types (FIG. 6A; silhouette score=0.17). This suggests that the diverse genomic contexts that a gene occupies are specific for different biomes, implying biome-specific gene semantics.

An ecologically important example of genomic “polysemy” (multiple meanings conferred by the same word) of methyl-coenzyme M reductase (MCR) complex was explored. The MCR complex is able to carry out a reversible reaction (Reaction 1 in FIG. 6B), whereby the forward reaction results in the production of methane (methanogenesis) while the reverse results in methane oxidation (methanotrophy). The McrA (methyl-coenzyme M reductase subunit alpha) protein in diverse lineages of ANME (ANaerobic MEthane oxidizing) and methanogenic archaeal genomes was first examined. These archaea are polyphyletic and occupy specific ecological niches. Notably, similar to how a semantic meaning of a word exists on a spectrum and a word can have multiple semantically appropriate meanings in a context, the MCR complex can confer different functions depending on the context. Previous reports demonstrate capacities of ANME (ANME-2 in particular) carrying out methanogenesis and methanogens conducting methane oxidation in specific growth conditions. The context-free ESM2 embedding of these proteins (FIG. 6B) shows little organization, with little separation between ANME-1 and ANME-2 McrA proteins. However, contextualized gLM embeddings (FIG. 6C) of the McrA proteins show distinct organization where ANME-1 McrA proteins form a tight cluster, while ANME-2 McrA proteins form a cluster with methanogens (silhouette score after contextualization: 0.24; before contextualization: 0.027). This organization reflects the phylogenetic relationships between the organisms that MerAs are found in, as well as the distinct operonic and structural divergence of MCR complexes in ANME-1 compared to those found in ANME-2 and methanogens. The preferred directionality in Reaction 1 in ANME-2 and some methanogens may be more dependent on thermodynamics.

It is also demonstrated that contextualized gLM embeddings are more suitable for determining the functional relationship between gene classes. Analogous to how the words “dog” and “cat” are closer in meaning relative to “dog” and “train”, a pattern was observed where Cas1- and Cas2-encoding genes appeared diffuse in multiple subclusters in context-free protein embedding space (FIG. 6D) but cluster in contextualized embedding space (FIG. 6E). This reflects their similarity in function (e.g. phage defense). This is also demonstrated in biosynthetic genes, where genes encoding lipopolysaccharide synthase (LPS) and polyketide synthase (PKS) cluster closer together in contextualized embedding space distinct from the Cas proteins (FIG. 6E). This pattern was quantitated with a higher silhouette score measuring phage defense and biosynthetic gene separation (gLM representation: 0.123±0.021, pLM representation: 0.085±0.007; paired t-test, t-statistic: 5.30, two-sided, p-value=0.0005, n=10). Contextualized protein embeddings are therefore able to capture relational properties akin to semantic information, where genes encoding proteins that are more similar in their function are found in similar genomic contexts.

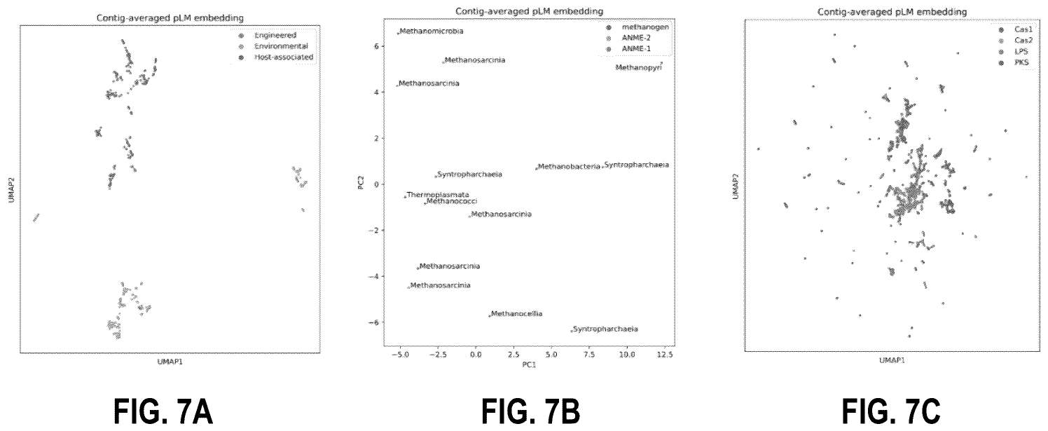

In order to quantify the information gained as a result of training a transformer on genomic contexts, clustering results in FIGS. 6A, 6C, and 6E were compared with clustering conducted on (sub)contig-averaged pLM embeddings (FIGS. 7A-7C). By mean-pooling pLM embeddings across a given subcontig, the context information could be summarized as a naive baseline. Most consistent clustering (higher silhouette scores) of gLM embeddings compared to contig-averaged pLM were observed for all three analyses (see FIGS. 7A-7C figure captions for values). These results demonstrate that the gLM transformer model learns representations that correlate with biological function, which are not captured by the naive baseline.

### Characterizing the Unknown

Metagenomic sequences feature many genes with unknown or generic functions, and some are so divergent that they do not contain sufficient sequence similarity to the annotated fraction of the database. In the dataset, of the 30.8M protein sequences, 19.8% could not be associated with any known annotation (see Methods), and 27.5% could not be associated with any known Pfam domains using a recent deep learning approach (ProtENN). Understanding the functional role of these proteins in their organismal and environmental contexts remains a major challenge because most of the organisms that house such proteins are difficult to culture and laboratory validation is often low-throughput. In microbial genomes, proteins conferring similar functions are found in similar genomic contexts due to selective pressures bestowed by functional relationships (e.g. protein-protein interactions, co-regulations) between genes. Based on this observation, it was posited that contextualization would provide richer information that pushes the distribution of unannotated genes closer to the distribution of annotated genes. The distributions of unannotated and annotated fractions of proteins in the dataset were compared using context-free pLM embeddings and contextualized gLM embeddings. A statistically significant lower divergence was found between distributions of unannotated and annotated genes in gLM embeddings compared to pLM embeddings (paired t-test of Kullback-Leibler divergences, t-test statistic=7.61, two-sided, p-value<1e-4, n=10; see Methods for sampling and metric calculation). This suggests a greater potential for using gLM embeddings to transfer validated knowledge in cultivable and well-studied strains (e.g. E. coli K-12) to the vastly uncultivated metagenomic sequence space. Genomic context, along with molecular structure and phylogeny, appear to be important information to abstract in order to effectively represent sequences such that hidden associations can be uncovered between the known and the unknown fractions of biology.

### Contextualization Improves Enzyme Function Prediction

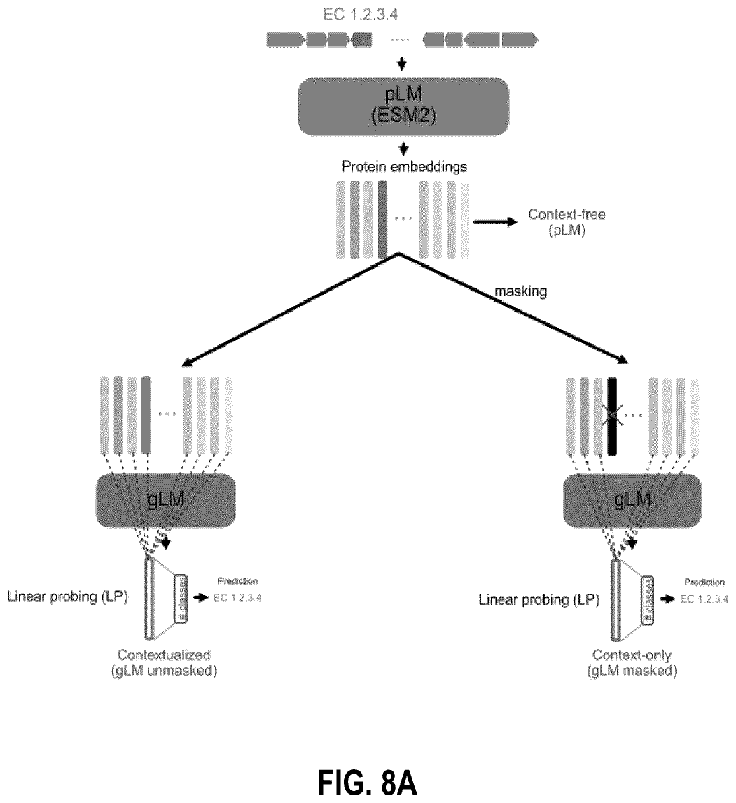

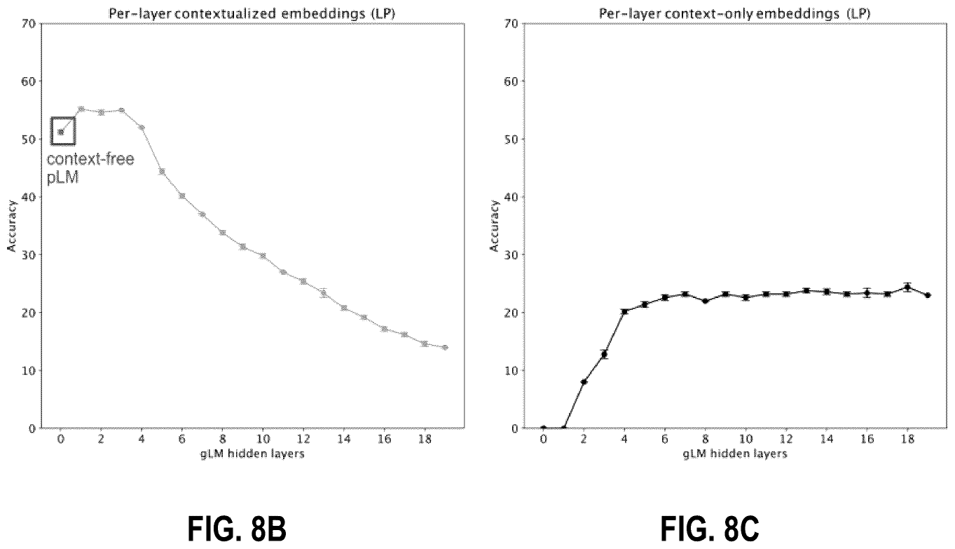

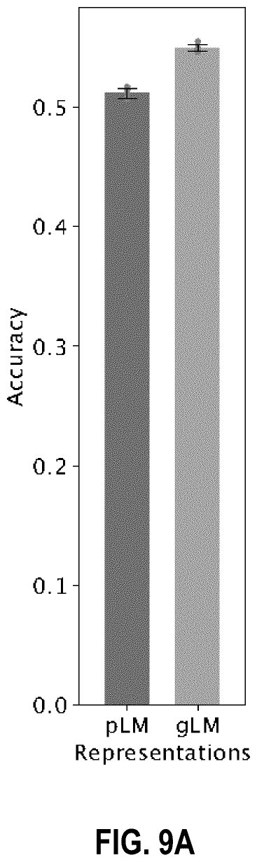

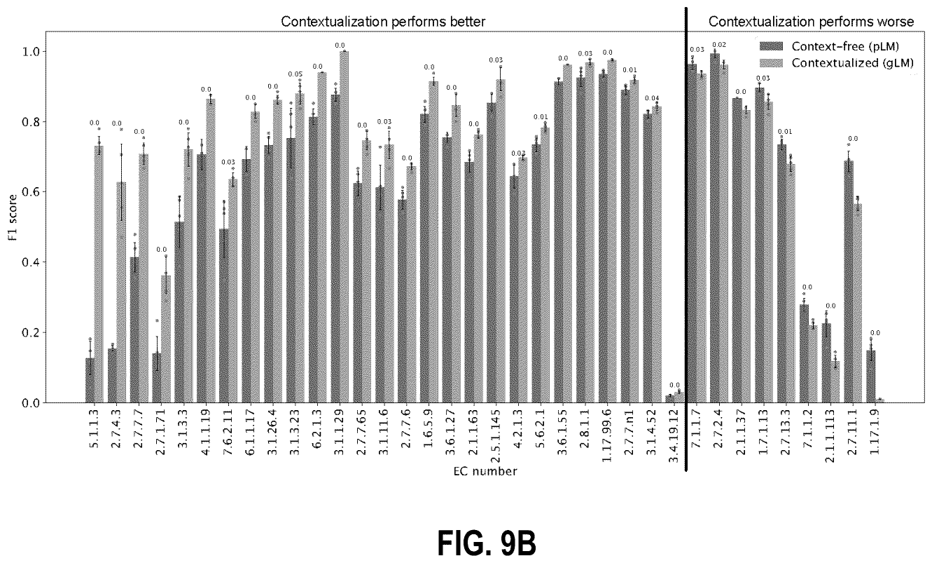

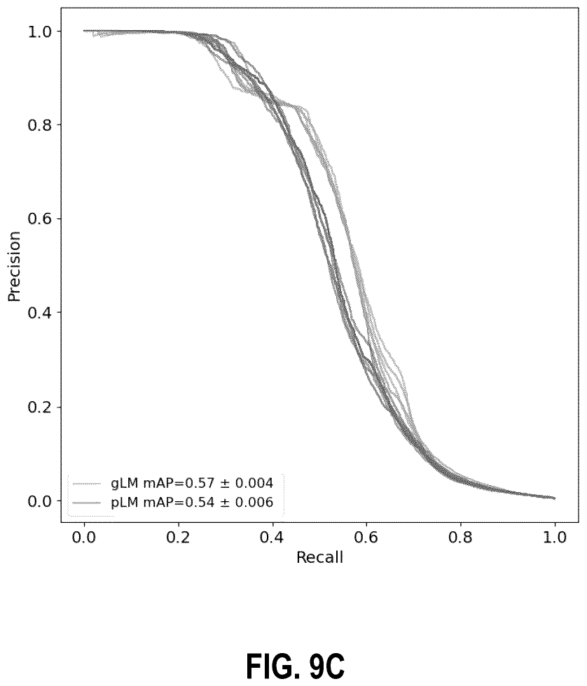

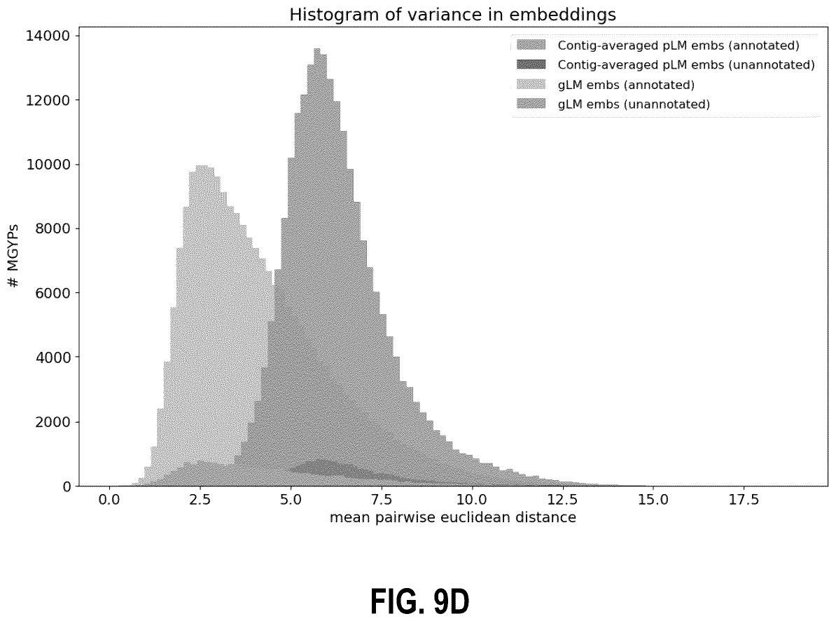

To test the hypothesis that the genomic context of proteins can be used to aid function prediction, how contextualization can improve the expressiveness of protein representations for enzyme function prediction was evaluated. First, a custom MGYP-EC dataset was generated where the train and test data were split at 30% sequence identity for each EC class (see Methods). Second, a linear probe (LP) was applied to compare the expressiveness of representations at each gLM layer, with and without masking the queried protein (FIGS. 8A-8C). By masking the queried protein, gLM's ability to learn functional information of a given protein, only from its genomic context, can be assessed without the propagation of information from the protein's pLM embeddings. It was observed that a large fraction of contextual information pertaining to enzymatic function is learned in the first six layers of gLM. It is also demonstrated that context information alone can be predictive of protein function, reaching up to 24.4±0.8% accuracy. In contrast, without masking, gLM can incorporate information present in the context with the original pLM information for each queried protein. An increase in expressivity of gLM embeddings also was observed in the shallower layers, with accuracy reaching up to 51.6±0.5% in the first hidden layer. This marks a 4.6±0.5% increase from context-free pLM prediction accuracy (FIG. 9A) and 5.5±1.0% increase in mean average precision (FIG. 9C) Thus, it is demonstrated that information that gLM learns from the context is orthogonal to information captured in pLM embedding. Diminishing expressivity in enzyme function information was also observed with deeper layers of gLM; this is consistent with previous examinations of LLMs, where deeper layers are specialized to the pretraining task (masked token prediction), and is consistent with previous examinations of LLMs, where the best-performing layer depends on the specific downstream tasks. Finally, to further examine the expressiveness of these representations, we compared per-class F1 score gains (FIG. 9B). Statistically significant differences in F1 scores (t-test, two-sided, Benjamini/Hochberg corrected p-value<0.05, n=5) were observed between the two models in 36 out of 73 EC classes with more than ten samples in the test set. Majority (27 out of 36) of the statistical differences resulted in improved F1 score in LP trained on gLM representations.

### Horizontal Transfer Frequency Corresponds to Genomic Context Embedding Variance

A key process that shapes microbial genome organization and evolution is horizontal gene transfer (HGT). The taxonomic range in which genes are distributed across the tree of life depends on their function and the selective advantage they incur in different environments. Relatively little is known about the specificity in the genomic region into which a gene gets transferred across phylogenetic distances. The variance of gLM embeddings was examined for proteins that occur at least one hundred times in the database. Variance of gLM-learned genomic contexts are calculated by taking a random sample of 100 occurrences and then calculating the mean pairwise distances between the hundred gLM embeddings. Such independent random sampling and distance calculation was conducted ten times per gene and then calculate the mean value. As a baseline, variance of subcontig-averaged pLM embeddings was calculated using the same sampling method, to compare the information learned from training gLM. These results show that gLM-learned genomic context variances have a longer right-hand tail (kurtosis=1.02, skew=1.08) compared to the contig-averaged pLM baseline that is more peaked (kurtosis=2.2, skew=1.05) (FIG. 9D). Notably, the most context-variant genes in the right tail of gLM-learned context variance distribution (orange) included phage genes and transposases, reflecting their ability to self-mobilize. Interestingly, no phage genes were found in the right-most tail of contig-averaged pLM embedding variance distribution (blue), although genes involved in transposition were found (Tables 4A-4B). gLM-learned genomic context variances can be used as a proxy for horizontal transfer frequencies and can be used to compare the fitness effects of the genomic context on the evolutionary trajectory (e.g. gene flow) of genes, as well as to identify undercharacterized and functional transposable elements.

Tables 4A-4B. Context-variant gene annotations.

TABLE 4A
List of top ten most context variant genes where variance
is calculated using gLM contextualized embeddings.

| | Mean embedding | |
|---|---|---|
| MGYP ID | variance | UniRef annotation |
| 815530568 | 18.87014775 | Uncharacterized protein n = 1 |
| 3385096772 | 17.70851124 | Tail fiber protein n = 20 |
| 2546832411 | 17.70269541 | Capsular exopolysaccharide family |
| | | n = 1 |
| 767058056 | 17.08193362 | Type I restriction enzyme |
| | | endonuclease subunit |
| 3381572716 | 16.44215407 | Ferrous iron transport protein B n = 11 |
| 2534762610 | 16.38703203 | None |
| 2733868861 | 16.21466745 | 30S ribosomal protein S18 n = 4 |
| 551790786 | 16.06301055 | 30S ribosomal protein S18 n = 285 |
| 3383952544 | 15.74879686 | CRISPR-associated endoribonuclease |
| | | Cas2 n = 88 |
| 832772145 | 15.62986097 | Ferrous iron transport protein A n = 6 |

TABLE 4B
List of top most context variant genes where
variance is calculated using contig-Averaged.

| | | | Mean | | |
|---|---|---|---|---|---|
| | | | embedding | | |
| 1. | MGYP ID | 2. | variance | 3. | UniRef annotation |
| 4. | 3384243084 | 5. | 18.43362238 | 6. | DDE_Tnp_1 domain- |
| | | | | | containing protein n = 137 |
| 7. | 943740416 | 8. | 17.77505976 | 9. | Phosphoribosylamine--glycine |
| | | | | | ligase n = 7 |
| 10. | 120256991 | 11. | 16.91703339 | 12. | Asparaginase n = 7 |
| 13. | 209907419 | 14. | 16.75686986 | 15. | DNA mismatch repair protein |
| | | | | | MutS n = 2 |
| 16. | 3380534673 | 17. | 16.67205026 | 18. | IS66-like element ISBf10 |
| | | | | | family transposase |
| 19. | 3384295730 | 20. | 16.44076785 | 21. | IS66-like element ISBf10 |
| | | | | | family transposase |
| 22. | 3381576767 | 23. | 16.23692972 | 24. | VOC domain-containing |
| | | | | | protein n = 96 |
| 25. | 3385319961 | 26. | 16.10454523 | 27. | Peptidase E n = 538 |
| 28. | 87924424 | 29. | 15.97918443 | 30. | 3-oxoacyl-[acyl-carrier- |
| | | | | | protein] synthase 3 |
| 31. | 997418675 | 32. | 15.62986097 | 33. | HNH homing endonuclease |
| | | | | | n = 2 |

### Transformer's Attention Captures Operons

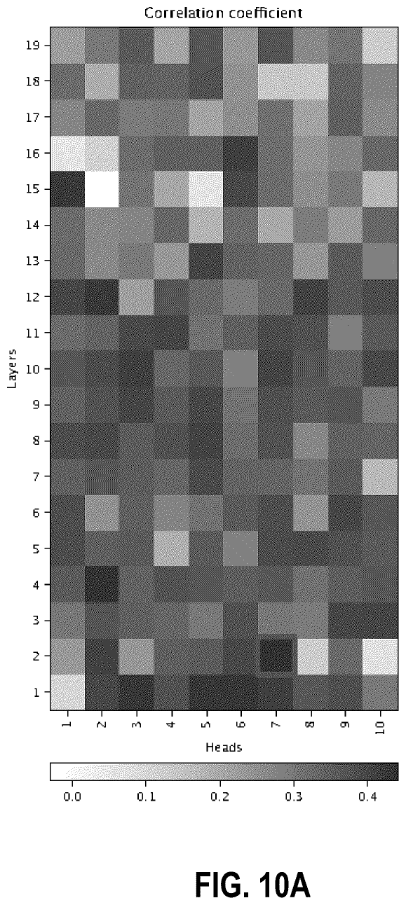

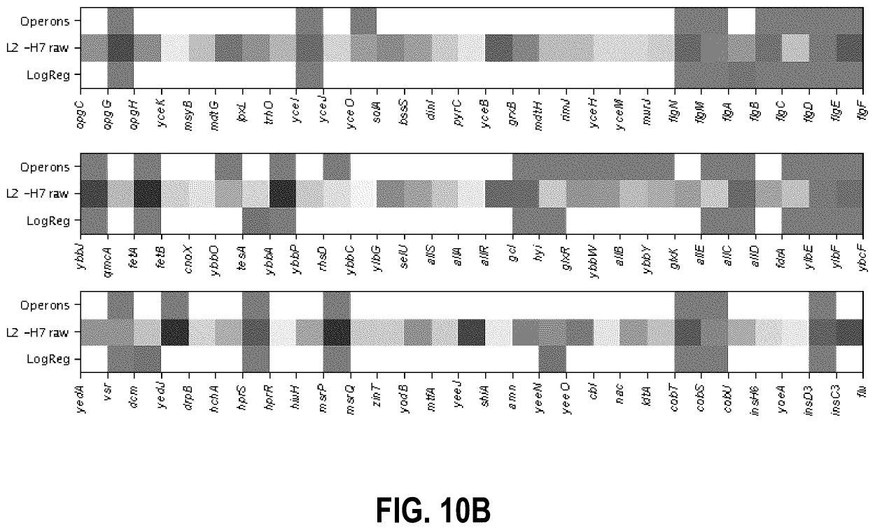

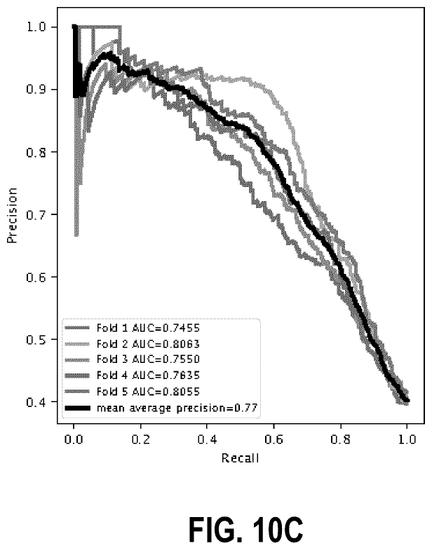

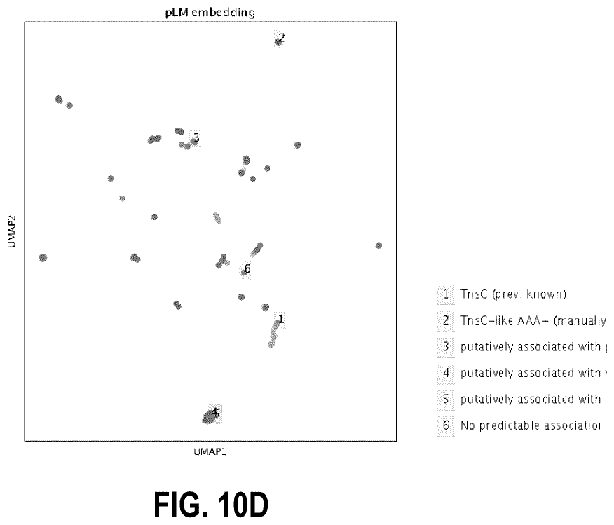

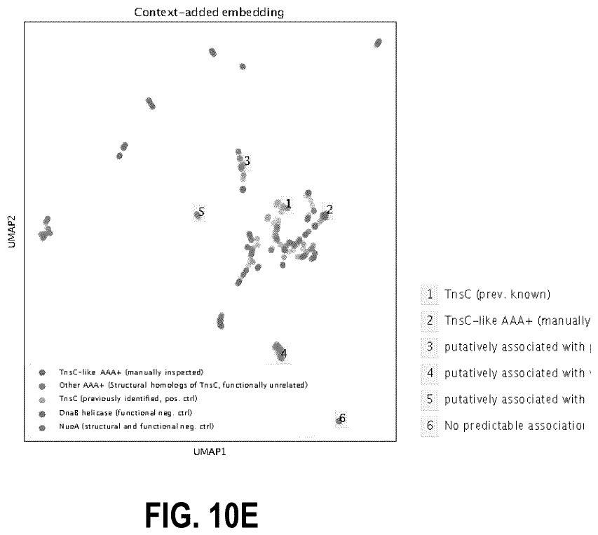

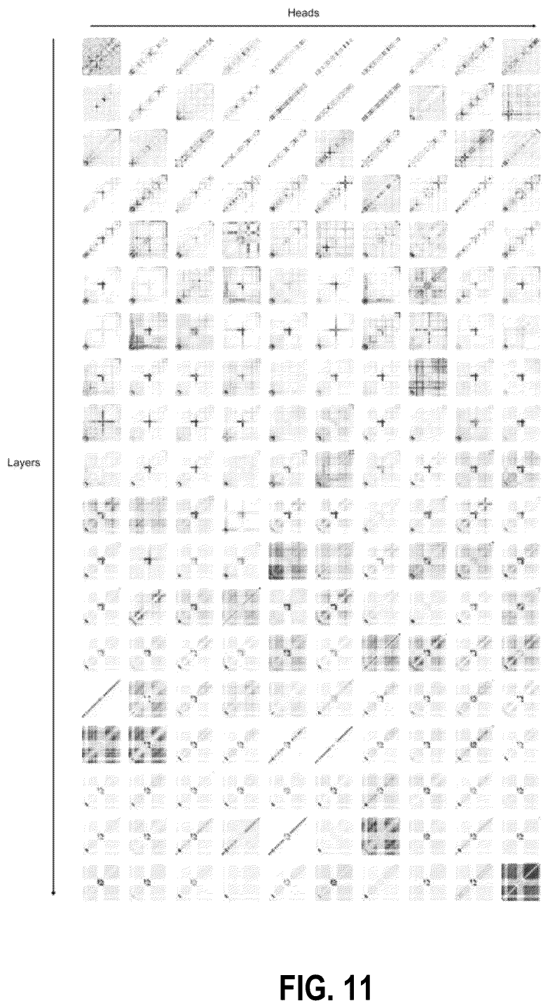

The transformer attention mechanism models pairwise interaction between different tokens in the input sequence. For the gLM presented herein, it was hypothesized that specific attention heads focus on learning operons, a “syntactic” feature pronounced in microbial genomes where multiple genes of related function are expressed as single polycistronic transcripts. Operons are prevalent in bacterial, archaeal and their viral genomes, while rare in eukaryotic genomes. The E. coli K-12 operon database consisting of 817 operons was used for validation. gLM contains 190 attention heads across 19 layers. It was found that heads in shallower layers correlated more with operons (FIG. 10A, FIG. 11, with raw attention scores in the 7th head of the 2th layer [L2-H7] linearly correlating with operons with 0.44 correlation coefficient (Pearson's rho, Bonferroni adjusted p-value<1E-5) (FIG. 10B). A logistic regression classifier (operon predictor) was further trained using all attention patterns across all heads. This classifier predicts the presence of an operonic relationship between a pair of neighboring proteins in a sequence with high precision (mean average precision=0.775±0.28, five-fold cross-validation) (FIG. 10C). This performance was baselined by training an operon predictor on the one-hot amino acid representation-based gLM ablation (mean average precision=0.426±0.015, five-fold cross-validation; Table 3), that learns from the orientation and co-occurrence information but cannot fully leverage rich representation of genes.

### Context Dependency of AAA+ Regulator Functions in Complex Genetic Systems

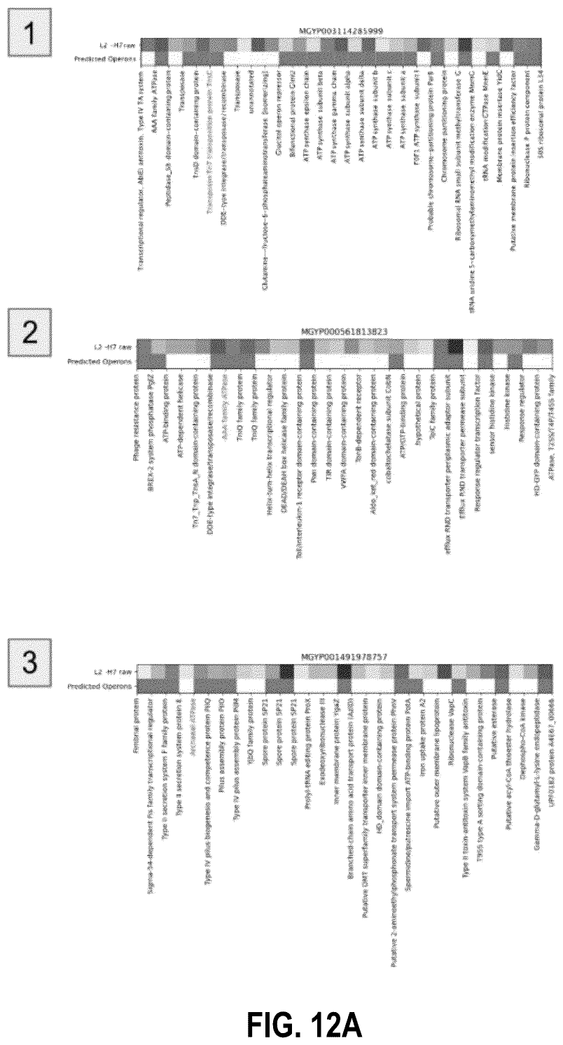

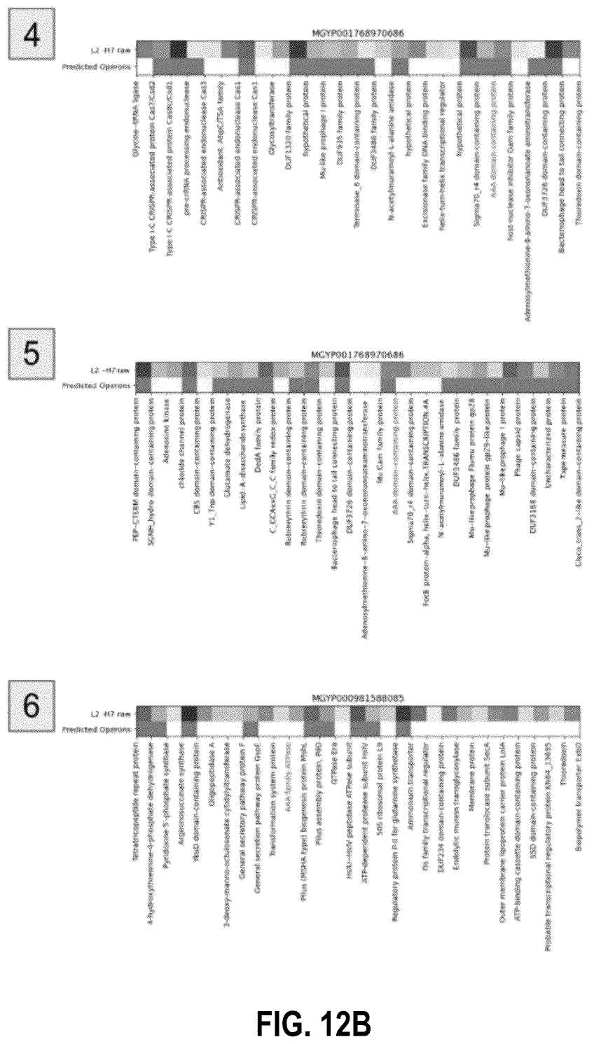

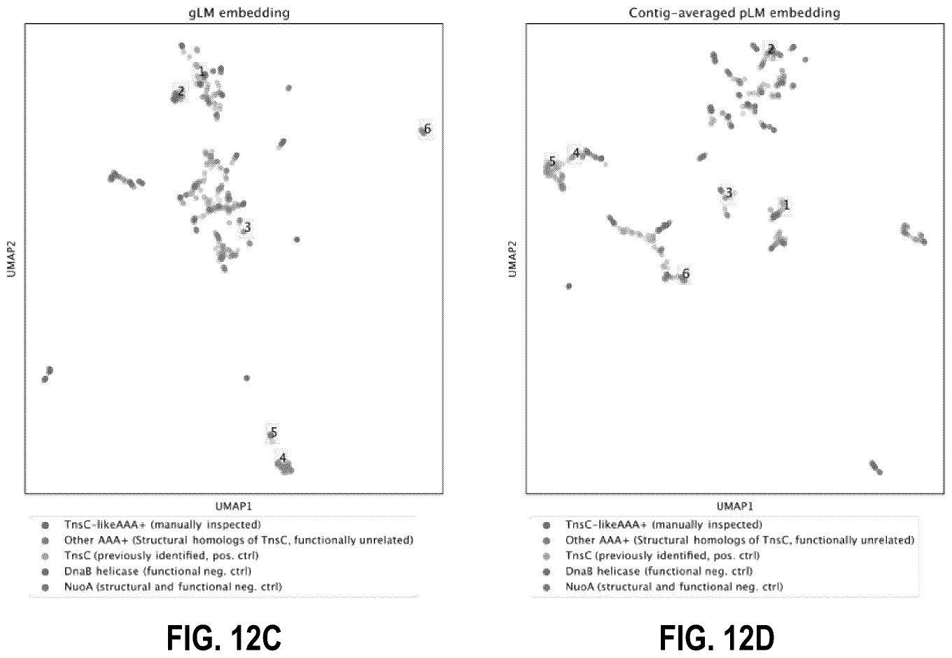

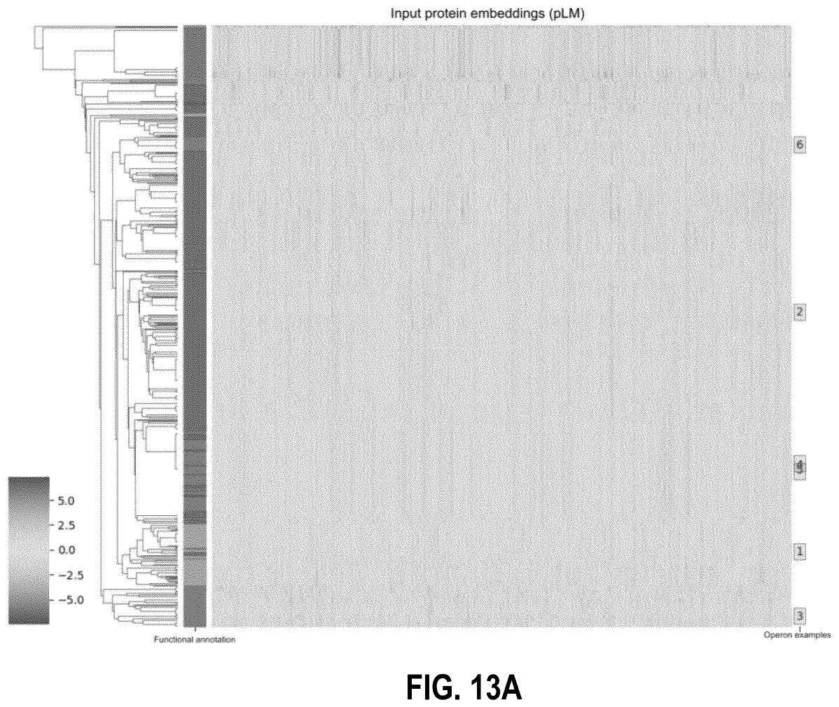

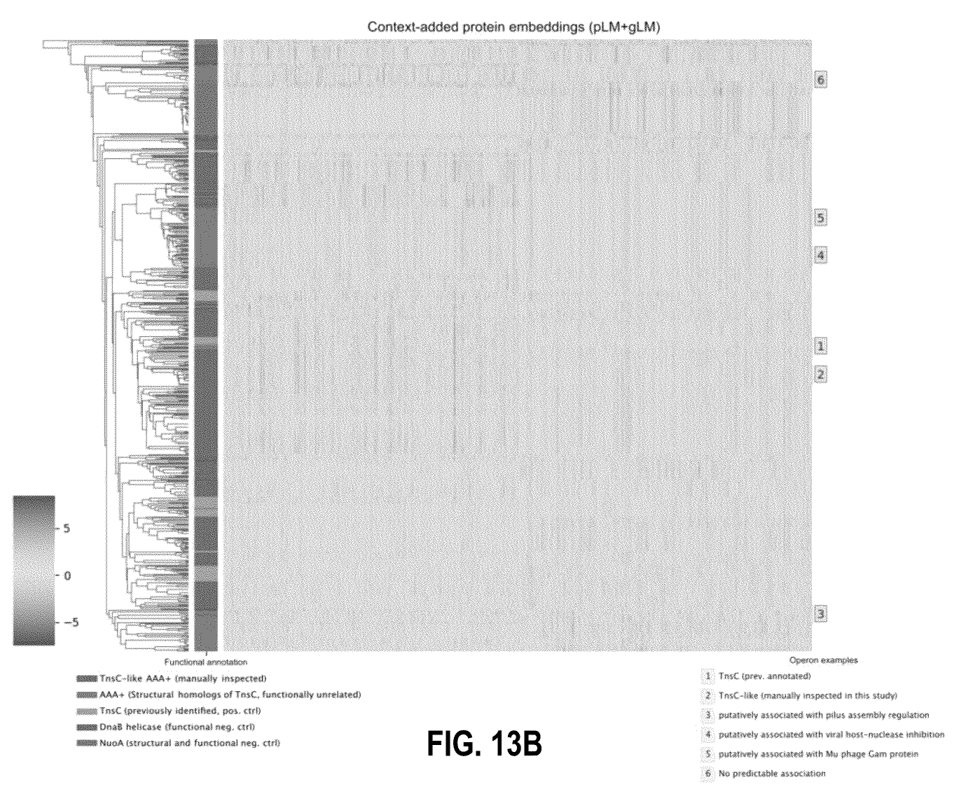

Understanding the functional role of a regulatory protein in an organism remains a challenging task because the same protein fold may carry out different functions depending on the context. For instance, AAA+ proteins (ATPases associated with diverse cellular activities) utilize the chemical energy from ATP hydrolysis to confer diverse mechanical cellular functions. However, AAA+ regulators can also play very different, broad functional roles depending on their cellular interacting partners from protein degradation and DNA replication to DNA transposition. One particularly interesting example is the TnsC protein, which regulates DNA insertion activity in Tn7-like transposon systems. Multiple bioinformatic efforts focused on discovery of previously uncharacterized transposons through metagenome search and sequence searches of assembled genomes aimed at identifying suitable homologs for genome-editing applications. In order to test whether the methods developed here could identify Tn7-like transposition systems as well as distinguish these from other functional contexts, the contextualized semantics of TnsC's structural homologs were explored in the MGnify database. Without contextualization, there appears no clustering with associated transposase activity (KL divergence ratio=1.03; sec Methods for calculation of this metric, FIG. 10D). However, with added contextualization, previously identified TnsC (orange) and manually inspected TnsC-like structural homolog (red, labeled “TnsC-like”) cluster together (KL divergence ratio=0.38; FIG. 10E; scc FIGS. 12C-12D for comparison with gLM-only and contig-averaged pLM baselines). This visualization was further validated using embedding distance-based clustering (FIGS. 13A-13B). Many structural homologs of TnsC were not involved in transposition and this is reflected in distinct clusters of gray data points away from known TnsC (oranges) and TnsC-like structural homologs (red) in FIG. 10D. These clusters represent diverse and context-dependent AAA+ regulation activity that cannot be predicted from neither structure nor raw sequence alone. An operonic relationship between these AAA+ regulators and their neighboring genes was predicted and many were found to be in operonic relationships with gene modules of diverse function including pilus assembly and viral host-nuclease inhibition (FIG. 12A). In some cases, queried AAA+ proteins did not appear to be in an operonic association with the neighboring proteins, suggesting some AAA+ proteins are less likely to be functionally associated with their neighbors than others (FIG. 12B, example 6). Using this example of AAA+ regulators, it is illustrated that combining the contextualized protein embeddings and attention-based operon interaction may provide an important avenue for exploring and characterizing the functional diversity of regulatory proteins.

### gLM Predicts Paralogy in Protein-Protein Interactions

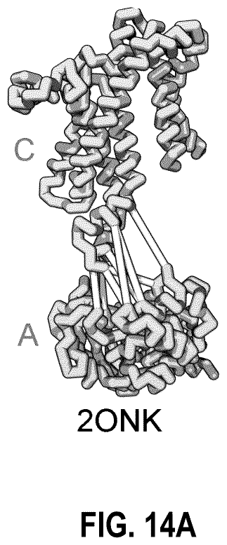

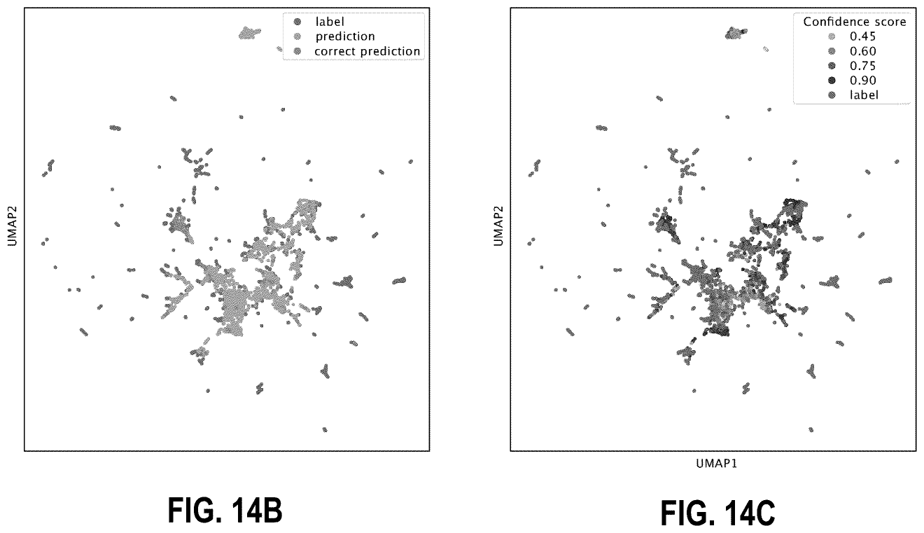

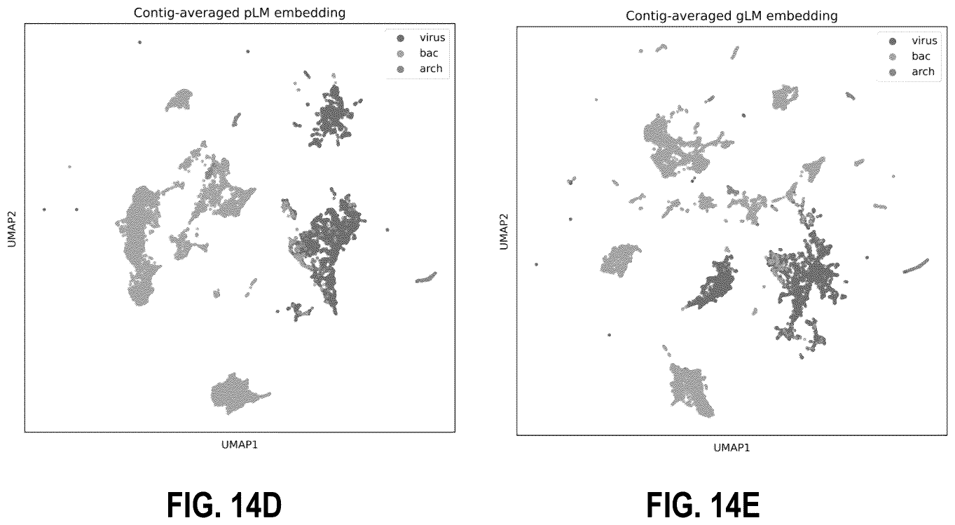

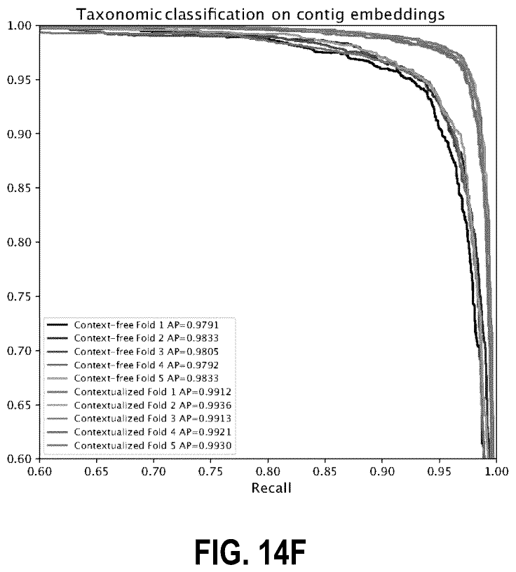

Proteins in an organism are found in complexes and interact physically with each other. Recent advances in protein-protein interaction (PPI) prediction and structural complex research has largely been guided by identifying interologs (conserved PPI across organisms) and co-evolutionary signals between residues. However, distinguishing paralogs from orthologs (otherwise known as the “Paralog matching” problem) in the expanding sequence dataset remains a computational challenge requiring queries across the entire database and/or phylogenetic profiling. In cases where multiple interacting pairs are found within an organism (e.g. histidine kinases (HK) and response regulators (RR)), prediction of interacting pairs is particularly difficult. It was reasoned that gLM, although not directly trained for this task, may have learned the relationships between paralogs versus orthologs. In order to test this capability, a well studied example of interacting paralogs (ModC and ModA, FIG. 14A) which form an ABC transporter complex was used. gLM was queried to predict the embedding of an interacting pair given no context except the protein sequence of either ModA or ModC. It was found that without any fine-tuning gLM performs at least an order of magnitude better than what is expected by random chance (see Methods). Specifically, for 398 out of 2700 interacting pairs, gLM makes predictions that belong to the same cluster (50% sequence identity, n=2100 clusters) as the true label, and in 73 pairs, the gLM predicts a label that is closest to the exact interacting pair (simulated random chance expected match=1.6 +1.01, n=10) (FIG. 14B). Importantly, when considering only very high confidence predictions (prediction likelihood>0.9, n=466), gLM is able to match paralogs with an increased accuracy of 25.1%. When paralogs are correctly paired, gLM is more confident about the prediction (average confidence for correct prediction=0.79, average confidence across all predictions=0.53), while less certain predictions are either out of distribution, or closer to the mean of labels (FIG. 14C).

### Contextualized Contig Embeddings and Potential for Transfer Learning

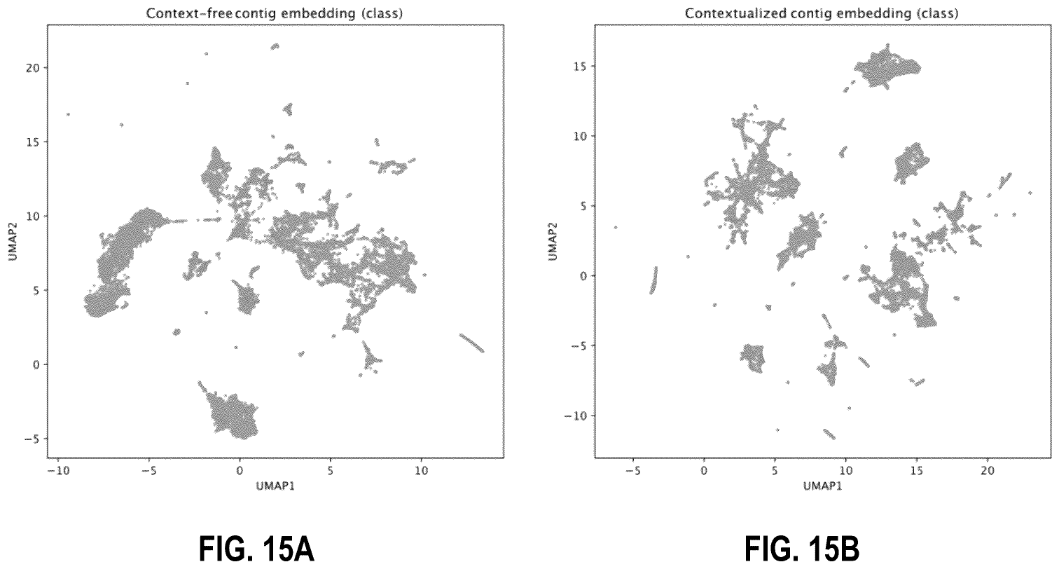

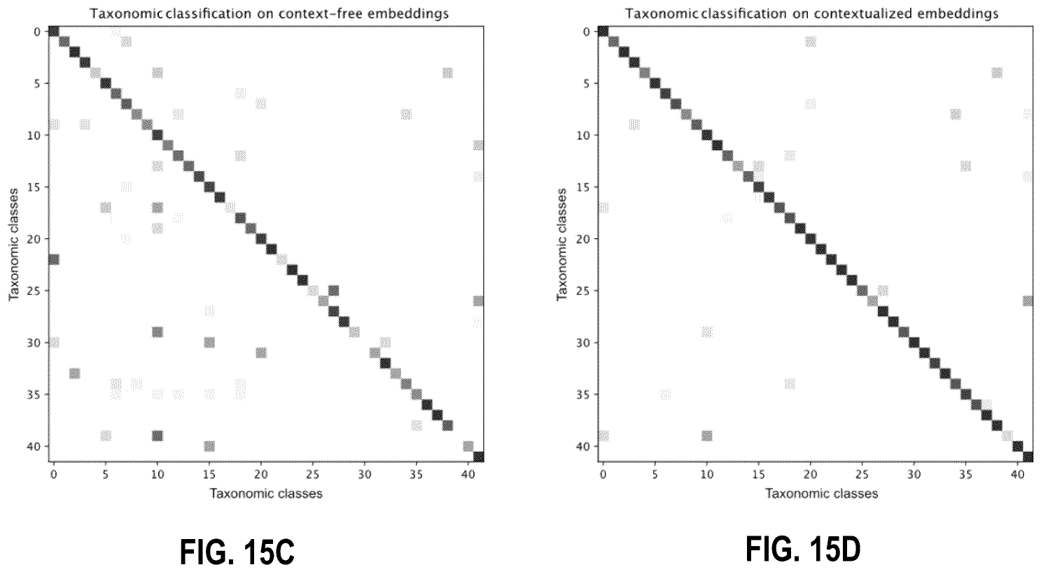

Contextualized protein embeddings encode the relationship between a specific protein and its genomic context, retaining the sequential information within a contig. It was hypothesized that this contextualization adds biologically meaningful information that can be utilized for further characterization of the multi-gene genomic contigs. Here, a contextualized contig embedding is defined as a mean-pooled hidden layer across all proteins in the subcontig, and a context-free contig embedding as mean-pooled ESM2 protein embeddings across the sequence (see methods). Both embeddings consist of 1280 features. This hypothesis was tested by examining each of these embeddings' ability to linearly distinguish viral sequences from bacterial and archaeal subcontigs. In metagenomic datasets, the taxonomic identity of assembled sequences must be inferred post-hoc, therefore the identification of viral sequences is conducted based on the presence of viral genes and viral genomic signatures. However, such classification task remains a challenge particularly for smaller contig fragments and less characterized viral sequences. Here, random 30-protein subcontigs were sampled from the representative bacterial and archaeal genome database and reference viral genomes in the NCBI and visualized their context-free contig embeddings (FIG. 14D) and contextualized contig embeddings (FIG. 14E). More separation and taxonomic clusters were observed at both domain-and class-levels (FIGS. 15A-15D), suggesting that taxonomic signature is enhanced by encoding the latent relationships between proteins. This is further validated by training a logistic regression classifier on context-free and contextualized contig embeddings for class-level taxonomy (FIGS. 15A-15B), where a statistically significant improvement in average precision is seen (FIG. 14F, see FIGS. 15C-15D for confusion matrices). This emphasizes the biological importance of a protein's relative position in the genome and its relationship with the genomic context, and further indicates that this information can be effectively encoded using gLM. Contextualized contig embeddings present opportunities for transfer learning beyond viral sequence prediction, such as improved metagenomically-assembled genome (MAG) binning and assembly correction.

### Methods

### Sequence Database

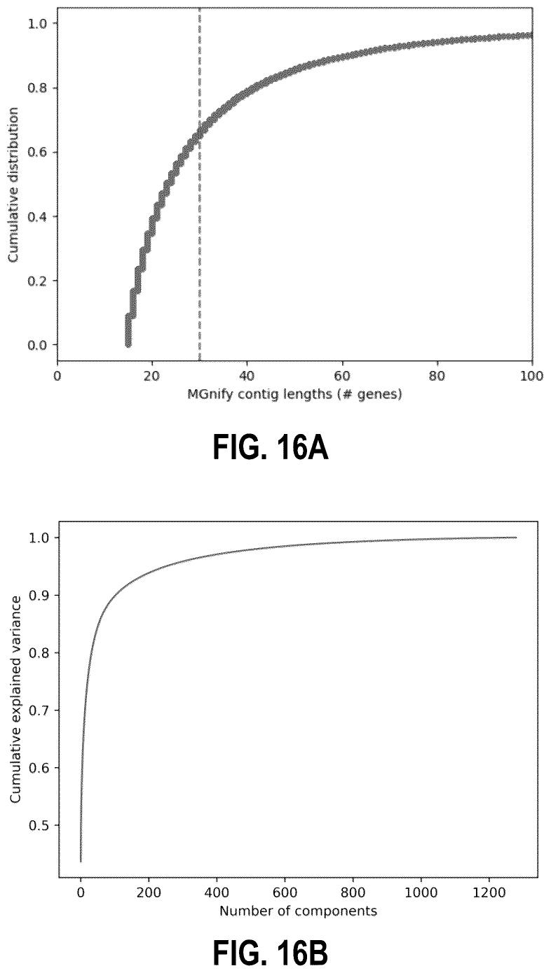

The genomic corpus was generated using the MGnify dataset (released 2022-05-06 and downloaded 2022-06-07). First, genomic contigs with greater than 30 genes were divided into 30 gene non-overlapping subcontigs resulting in a total of 7,324,684 subcontigs with lengths between 15 and 30 genes (subcontigs<15 genes in length were removed from the dataset). 30 was chosen as maximum context length because while longer context results in higher modeling performance, 67% of the raw MGnify contigs with >15 genes were of =<30 genes in length (FIG. 16A), and therefore increasing the context length beyond 30 would have resulted in many examples with padding (reduced computational efficiency). Each gene in the subcontig was mapped to a representative protein sequence (representative MGYP) using mmseqs/linclust, with coverage and sequence identity thresholds set at 90% (pre-computed in the MGnify database), resulting in a total of 30,800,563 representative MGYPs. Each representative MGYP was represented by a 1280-feature protein embedding, generated by mean-pooling the last hidden layer of the ESM2 “esm2_t33_650M_UR50D” model. Due to the memory limitation in computing embeddings for very long sequences, 116 of the MGYP sequences longer than 12290 amino acids were truncated to 12290 amino acids. ESM2 embeddings were normalized (by subtracting the mean of each feature and dividing by its standard deviation) and clipped such that all features range from −10 to 10, to improve training stability. A small fraction (0.4%) of the genes could not be mapped to a representative MGYP and therefore the corresponding sequence information could not be retrieved from the MGnify server; these sequences were assigned a 1280 feature vector of ones. For each gene in the sub-sequence, a gene orientation feature was added to the standardized MGYP protein embedding, where 0.5 denotes “forward” orientation relative to the direction of sequencing, and −0.5 denotes “reverse” orientation. Thus, each gene was represented by a 1281 feature vector in the corpus. gLM architecture and training

gLM was built on the huggingface implementation of the ROBERTa transformer architecture. gLM consisted of 19 layers with hidden size 1280 and ten attention heads per layer, with relative position embedding (“relative_key_query”). For training, 15% of the tokens (genes) in the sequence (subcontig) were randomly masked to a value of-1. The model was then tasked with the objective of predicting the label of the masked token, where the label consists of a 100-feature vector that consists of the PCA whitened 99 principal components (explained variance=89.7%. FIG. 16B) of the corresponding ESM2 protein embedding concatenated with its orientation feature. Reduced dimensionality of labels using PCA increased the stability of training. Specifically, gLM projects the last hidden state of the model into four 100-feature vectors and four corresponding likelihood values using a linear layer. Total loss is calculated using Equation 1.

The closest prediction is defined as the prediction that is closest to the label, computed by L2 distance. α=1e-4. gLM was trained in half precision with batch size 3000 with distributed data parallelization on four NVIDIA A100 GPUs over 1,296,960 steps (560 epochs) including 5000 warm-up steps to reach a learning rate of 1e-4 with AdamW optimizer.

### Performance Metric and Validation

In order to evaluate the model quality and its generalizability beyond the training dataset, a pseudo-accuracy metric was used, where a prediction to be “correct” was deemed if it was closest in Euclidean distance to the label of the masked gene relative to the other genes in the subcontig. Pseudo-accuracy calculation is described in Equation 2.

The metric and subsequent analyses was validated on the best annotated genome to date: E. coli K-12. In order to remove as many E. coli K-12 like subcontigs from the training dataset, 5.2% of the subcontigs in which more than half of the genes were >70% similar (calculated using mmseqs2 search) in amino acid sequence to E. coli K-12 genes were removed. The pseudo accuracy metric was validated by calculating the absolute accuracy on the E. coli K-12 genome for which each gene was masked sequentially (Equation 3).

$$absolute accuracy = \frac{\begin{matrix}\# count ( argmin ( dist ( prediction , all genes \\ in E . coli K - 12 ) ) == index ( masked gene ) )\end{matrix}}{\# genes in E . coli K - 12} \qquad ( Equation 3 )$$

### Contextualized Embedding Calculation and Visualization

Contextualized protein embedding of a gene is calculated by first inputting a 15-30 gene subcontig containing the gene of interest, and then running inference on the subcontig using the trained gLM without masking. The last hidden layer of the model corresponding to the gene was then used as the embedding consisting of 1280 features.

### Gene Annotation

Genes were annotated using Diamond v2.0.7.145 against the UniRef90 database with an e-value cut-off 1E-5. Genes were labeled as “unannotated” if either 1) no match was found in the UniRef90 database, or 2) the match was annotated with following keywords: “unannotated”, “uncharacterized”, “hypothetical”, “DUF”(domain of unknown function).

### McrA Protein Analysis

McrA protein encoding Methanogens and ANME genomes were selected from the accession ID list found in the supplement of Shao et al. subcontigs containing mcrA were extracted with at most 15 genes before and after mcrA. The context-free and contextualized embeddings of McrA were calculated using the ESM2 and gLM respectively.

Distributions of Unannotated and Annotated Embeddings

Distributions of unannotated and annotated embeddings in the database were compared using Kullback-Leibler (KL) divergence analysis. First, ten random samples of 10,000 subcontigs from the MGnify corpus. pLM and gLM embeddings of the genes were calculated using mean-pooled last hidden layer of ESM2 embeddings and mean-pooled last hidden layer of gLM respectively. Outliers were removed using Mahalanobis distance and a chi-squared threshold of 0.975. pLM and gLM embedding dimensions were reduced to 256 principal components (91.9±1.72% and 80.1±6.89% total variances explained respectively). KL divergence was calculated using the following Equation 4.

$${D}_{K L} ( P \vert \vert Q ) = \frac{1}{2} ( tr ( {\sum }_{1}^{- 1} {\sum }_{0} ) - k + {( {μ}_{1} - {μ}_{0} )}^{T} {\sum }_{1}^{- 1} ( {μ}_{1} - {μ}_{0} ) + ln ( \frac{det {\sum }_{1}}{det 0} ) ) \qquad ( Equation 4 )$$

where P corresponds to the distribution of unannotated genes and Q corresponds to the distribution of annotated genes, with μ₀, μ₁ respectively as means and Σ₀, Σ₁ respectively as covariance matrices. The significance of the KL divergence differences between pLM and gLM embeddings is calculated using a paired t-test across the ten samples.

### Enzyme Commission Number Prediction

Custom MGYP-Enzyme Commission (MGYP-EC) dataset was created by first searching (mmseqs2 with default setting) MGYPs against the “split30.csv” dataset previously used to train CLEAN. “split30.csv” dataset consists of EC numbers assigned to UniProt sequences clustered at 30% identity. Only MGYP hits with >70% sequences to “split30.csv” were considered and MGYPs with multiple hits with >70% similarity were removed. Test split was selected by randomly selecting 10% of “split30.csv” UniProt IDs in each EC category that map to MGYPs. EC categories with less than four distinct UniProt IDs with MGYP mapping were removed from the dataset, resulting in 253 EC categories. The train set consisted of MGnify subcontigs in the corpus that contained at least one the 27936 MGYPs mapping to 1878 UniProt IDs. The test set consisted of randomly selected MGnify subcontig containing each of 4441 MGYPs mapping to 344 UniProt IDs. pLM (context-free) embeddings were calculated for each of MGYP with EC number assignment by mean-pooling the last hidden layer of its ESM2 embedding. Masked (context-only) gLM embeddings were calculated for each of the 19 layers by running inference on subcontigs with masks at the positions of MGYPs with EC number assignment and subsequently extracting per-layer hidden representations for masked positions. gLM (contextualized) embeddings were calculated also for each layer by running inference without masking and subsequently extracting per-layer hidden representations for MGYPs with EC number assignments. Linear probing was conducted for these embeddings with a single linear layer. Linear probes were trained with early stopping (patience=10, github.com/Bjarten/carly-stopping-pytorch/blob/master/pytorchtools.py) and batch size=5000, and training results were replicated five times with random seeds to calculate error ranges.

### Variance of Contextualized Protein Embedding Analysis

Contextualized protein embeddings are generated at inference time. Variances of contextualized protein embeddings were calculated for MGYPs that occur at least 100 times in the dataset, excluding the occurrences at the edges of the subcontig (first or last token). For each such MGYP, 10 random independent samples consisting of 100 occurrences were taken and the mean pairwise euclidean distances between the contextualized embeddings were calculated. To assess the role gLM plays in contextualization, the above sampling method was used to calculate the variance of contig-averaged pLM embeddings (pLM embeddings mean-pooled across the contig) for each MGYP that occurs at least 100 times in the dataset.

### Attention Analysis

Attention heads (n=190) were extracted by running inference on unmasked subcontigs, and the raw attention weights were subsequently symmetrized. E. coli K-12 RegulonDB was used to probe heads with attention patterns that correspond the most with operons. Pearson's correlation between symmetrized raw attentions and operons were calculated for each head. A logistic regression classifier was trained that predicts whether two neighboring genes belong to the same operon based on the attention weights across all attention heads corresponding to the gene pair.

### TnsC Structural Homolog Analysis

TnsC structural homologs were identified by searching ShCAST TnsC (PDB 7M99 chain H) against the MGYP database using Foldseek on ESM Atlas (https://esmatlas.com/). The contigs containing these homologs in the MGnify database were used to calculate the contextualized protein embeddings of the identified structural homologs. Contigs with less than 15 genes were excluded from the analysis. Contigs encoding proteins that were previously identified as “TnsC” using the UniRef90 database (see Gene annotation methods section above) were included in the database. “TnsC-like” contigs were manually annotated based on the presence of transposase genes (TnsB) and TniQ. Fifty random examples of MGnify contigs containing MGYPs annotated as NuoA and DnaB were added as negative controls for the UMAP visualization. KL divergence ratios were calculated using the following Equation 5.

$${D}_{K L} ( B \vert \vert A ) {D}_{K L} ( C \vert \vert A ) \qquad ( Equation 5 )$$

where A is the distribution of representations of known TnsC, B is the distribution of representations of manually curated TnsC-like AAA+ regulators, C is the distribution of representations of other AAA+ regulators that are functionally unrelated structural homologs of known TnsC. Therefore, this metric ranges from 0 to 1, where a lower ratio represents increased ability to functionally discriminate distribution of B from C relative to A. KL divergence was calculated using the same formula as in the methods section Distributions of unannotated and annotated embeddings, except with 20 principal components that explained >85% of variances across all embeddings.

### Paralogy and Orthology Analysis

UniProt IDs from ABC transporter ModA and ModC protein interacting paralog pairs (n=4823) were previously identified by Ovchinnikov et al48 and were downloaded from gremlin.bakerlab.org/cplx.php?uni_a=2ONK_A&uni_b=2ONK_C and subsequently used to download raw protein sequences from the UniProt server. Only pairs (n=2700) where both raw sequences were available for download, and where the UniProt ID differed by one (indicating adjacent positioning in the reference genome) were selected for subsequent analyses. Test contigs were constructed consisting of three genes, where first and third genes are masked, and the second gene encodes one of the pair in forward direction. gLM was then queried to predict the two neighboring masked genes, and considered the prediction to be correct if either of the proteins closest to masked genes's highest confidence prediction in embedding space belongs to the same sequence cluster as the interacting protein (50% amino acid sequence identity, calculated using CD-HIT v4.6). Random chance correct prediction rate (1.6±1.0 was simulated using 1000 iterations of random predictions generated within the standard normal distribution and performing the same operation as above to compute the rate of correct predictions.

### Taxonomic Analysis and Visualization

4551 bacterial and archeal representative genomes and 11660 reference viral genomes were downloaded from the RefSeq database (ftp.ncbi.nlm.nih.gov/genomes/refseq) on 12 Feb. 2023. A random 30-gene subcontig is chosen and encoded using ESM2, which then were subsequently concatenated with an orientation vector and then used as input for the trained gLM. The last hidden layer was mean-pooled across the sequence to retrieve 1280-feature contextualized contig embeddings. The ESM2 protein embeddings were also mean-pooled across the sequence to retrieve 1280-feature context-free contig embeddings. A logistic regression classifier was trained to predict the class-level taxonomy of subcontigs and evaluated the performance using stratified k-fold cross-validation (k=5).

### UMAP Visualization and Statistical Tests

All UMAP dimensionality reductions calculated with following parameters: n_neighbors=15, min_dist=0.1. Silhouette scores were calculated using the sklearn package using the default setting with euclidean distance metric.

### Computer Implementation

An illustrative implementation of a computer system 1700 that may be used in connection with any of the embodiments of the technology described herein (e.g., such as the processes of FIG. 3A and 3B) is shown in FIG. 17. The computer system 1700 includes one or more processors 1710 and one or more articles of manufacture that comprise non-transitory computer-readable storage media (e.g., memory 1720 and one or more non-volatile storage media 1730). The processor 1710 may control writing data to and reading data from the memory 1720 and the non-volatile storage media 1730 in any suitable manner, as the aspects of the technology described herein are not limited to any particular techniques for writing or reading data. To perform any of the functionality described herein, the processor 1710 may execute one or more processor-executable instructions stored in one or more non-transitory computer-readable storage media (e.g., the memory 1720), which may serve as non-transitory computer-readable storage media storing processor-executable instructions for execution by the processor 1710.

Computing system 1700 may include a network input/output (I/O) interface 1740 via which the computing device may communicate with other computing devices. Such computing devices may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks, wired networks or fiber optic networks.

Computing system 1700 may also include one or more user I/O interfaces 1750, via which the computing device may provide output to and receive input from a user. The user I/O interfaces may include devices such as a keyboard, a mouse, a microphone, a display device (e.g., a monitor or touch screen), speakers, a camera, and/or various other types of I/O devices.

Further, it should be appreciated that a computer may be embodied in any of a number of forms, such as a rack-mounted computer, a desktop computer, a laptop computer, or a tablet computer, as examples. Additionally, a computer may be embedded in a device not generally regarded as a computer but with suitable processing capabilities, including a Personal Digital Assistant (PDA), a smartphone, a tablet, or any other suitable portable or fixed electronic device.

The above-described embodiments can be implemented in any of numerous ways. For example, the embodiments may be implemented using hardware, software, or a combination thereof. When implemented in software, the software code can be executed on any suitable processor (e.g., a microprocessor) or collection of processors, whether provided in a single computing device or distributed among multiple computing devices. It should be appreciated that any component or collection of components that perform the functions described above can be generically considered as one or more controllers that control the above-described functions. The one or more controllers can be implemented in numerous ways, such as with dedicated hardware, or with general purpose hardware (e.g., one or more processors) that is programmed using microcode or software to perform the functions recited above.

In this respect, it should be appreciated that one implementation of the embodiments described herein comprises at least one computer-readable storage medium (e.g., RAM, ROM, EEPROM, flash memory or other memory technology, CD-ROM, digital versatile disks (DVD) or other optical disk storage, magnetic cassettes, magnetic tape, magnetic disk storage or other magnetic storage devices, or other tangible, non-transitory computer-readable storage medium) encoded with a computer program (i.e., a plurality of executable instructions) that, when executed on one or more processors, performs the above-described functions of one or more embodiments. The computer-readable medium may be transportable such that the program stored thereon can be loaded onto any computing device to implement aspects of the techniques described herein. In addition, it should be appreciated that the reference to a computer program which, when executed, performs any of the above-described functions, is not limited to an application program running on a host computer. Rather, the terms computer program and software are used herein in a generic sense to reference any type of computer code (e.g., application software, firmware, microcode, or any other form of computer instruction) that can be employed to program one or more processors to implement aspects of the techniques described herein.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present disclosure need not reside on a single computer or processor but may be distributed in a modular fashion among a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

The foregoing description of implementations provides illustration and description but is not intended to be exhaustive or to limit the implementations to the precise form disclosed. Modifications and variations are possible in light of the above teachings or may be acquired from practice of the implementations. In other implementations the methods depicted in these figures may include fewer operations, different operations, differently ordered operations, and/or additional operations. Further, non-dependent blocks may be performed in parallel.

It will be apparent that example aspects, as described above, may be implemented in many different forms of software, firmware, and hardware in the implementations illustrated in the figures.

Having thus described several aspects and embodiments of the technology set forth in the disclosure, it is to be appreciated that various alterations, modifications, and improvements will readily occur to those skilled in the art. Such alterations, modifications, and improvements are intended to be within the spirit and scope of the technology described herein. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize or be able to ascertain using no more than routine experimentation many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as an example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as an example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The terms “approximately,” “substantially,” and “about” may be used to mean within ±20% of a target value in some embodiments, within ±10% of a target value in some embodiments, within ±5% of a target value in some embodiments, within ±2% of a target value in some embodiments. The terms “approximately,” “substantially,” and “about” may include the target value.

What is claimed is:

1. A method for generating a contextual embedding of a gene, the method comprising:
using at least one computer hardware processor to perform:
obtaining information specifying genomic context of the gene, the genomic context containing a plurality of genes including the gene, the information containing gene sequences for the plurality of genes;
encoding the information specifying the genomic context to obtain an initial encoding of the genomic context, the encoding comprising:
mapping the gene sequences to protein sequences; and
encoding the protein sequences using a trained protein language model (pLM) to obtain the initial encoding of the genomic context; and
processing the initial encoding of the genomic context with a genomic language model (gLM) to obtain the contextual embedding of the gene.

2. The method of claim 1, wherein the genomic context is a gene subcontig containing the plurality of genes.

3. The method of claim 1, wherein the genomic context consists of 10-50 genes.

4. The method of claim 1, wherein mapping the gene sequences to protein sequences comprises identifying for each of the gene sequences a representative protein sequence.

5. The method of claim 1, wherein the pLM is an ESM2 protein language model.

6. The method of claim 1, wherein the genomic context comprises the plurality of genes and a plurality of intergenic regions, the information containing intergenic sequences for the plurality of intergenic regions, and wherein encoding the information specifying the genomic context further comprises:
encoding the protein sequences and the intergenic sequences to obtain the initial encoding of the genomic context, the initial encoding comprising representations of the protein sequences and representations of the intergenic sequences.

7. The method of claim 6, wherein encoding the protein sequences and the intergenic sequences to obtain the initial encoding of the genomic context comprises:
encoding the protein sequences using the trained pLM to obtain the representations of the protein sequences; and
encoding the intergenic sequences using a trained intergenic sequence model to obtain the representations of the intergenic sequences.

8. The method of claim 1,
wherein the genomic context includes K genes and the information includes K gene sequences;
wherein mapping the gene sequences to protein sequences comprises mapping the K gene sequences to K protein sequences; and
wherein encoding the protein sequences comprises encoding each of the protein sequences as an N-dimensional vector such that the initial encoding of the genomic context comprises K N-dimensional vectors.

9. The method of claim 8, wherein K is between 15 and 30, inclusive, and wherein N is between 800 and 1600.

10. The method of claim 1, wherein the genomic language model comprises a multi-layer transformer model.

11. The method of claim 10, wherein the contextual embedding of the gene is obtained from hidden states of the genomic language model.

12. The method of claim 11, wherein the contextual embedding of the gene is obtained from the last hidden states of the genomic language model.

13. The method of claim 10, wherein the genomic language model comprises multiple hidden layers and multiple attention heads per layer.

14. The method of claim 1, further comprising:
using the contextual embedding of the gene to identify a putative function to a protein corresponding to the gene.

15. The method of claim 14, wherein using the contextual embedding to identify the putative function comprises comparing the contextual embedding of the gene to contextual embeddings of other genes whose proteins have functional annotations.

16. The method of claim 1, further comprising using the context embedding of the gene for annotation transfer.

17. The method of claim 1, wherein the gene is a microbial gene.

18. The method of claim 1, further comprising: obtaining one or more attention mappings from the gLM.

19. A system, comprising:
at least one computer hardware processor; and
at least one non-transitory computer-readable storage medium storing processor-executable instructions that, when executed by the at least one computer hardware processor, cause the at least one computer hardware processor to perform a method for generating a contextual embedding of a gene, the method comprising:
obtaining information specifying genomic context of the gene, the genomic context containing a plurality of genes including the gene, the information containing gene sequences for the plurality of genes;
encoding the information specifying the genomic context to obtain an initial encoding of the genomic context, the encoding comprising:
mapping the gene sequences to protein sequences; and
encoding the protein sequences using a trained protein language model (pLM) to obtain the initial encoding of the genomic context; and
processing the initial encoding of the genomic context with a genomic language model (gLM) to obtain the contextual embedding of the gene.

20. At least one non-transitory computer-readable storage medium storing processor-executable instructions that, when executed by at least one computer hardware processor, cause the at least one computer hardware processor to perform a method for generating a contextual embedding of a gene, the method comprising:
obtaining information specifying genomic context of the gene, the genomic context containing a plurality of genes including the gene, the information containing gene sequences for the plurality of genes;
encoding the information specifying the genomic context to obtain an initial encoding of the genomic context, the encoding comprising:
mapping the gene sequences to protein sequences; and
encoding the protein sequences using a trained protein language model (pLM) to obtain the initial encoding of the genomic context; and
processing the initial encoding of the genomic context with a genomic language model (gLM) to obtain the contextual embedding of the gene.
